SEQUENCE LISTING (8 sequences)

SEQ ID NO: 1 - DNA, 22 bp, Artificial Sequence
  Primer Sequence
atgtaagctcctggggattcac

SEQ ID NO: 2 - DNA, 22 bp, Artificial Sequence
  Primer Sequence
aagtaagtgactggggtgagcg

SEQ ID NO: 3 - DNA, 34 bp, Artificial Sequence
  Primer Sequence
ggggttctgagaggaagaccccccattgtaccac

SEQ ID NO: 4 - DNA, 44 bp, Artificial Sequence
  Primer Sequence
tcctacgggaggcagcagtggactaccagggtatctatcctgtt

SEQ ID NO: 5 - DNA, 43 bp, Artificial Sequence
  Primer Sequence
aaaggaagattaataccgcataaatcttgcgaccgtactcccc

SEQ ID NO: 6 - DNA, 45 bp, Artificial Sequence
  Primer Sequence
cccttattgttagttgccatcattactcgttgtacttcccattgt

SEQ ID NO: 7 - DNA, 43 bp, Artificial Sequence
  Primer Sequence
ggaatcttccacaatggacgcgctttacgcccaataaatccgg

SEQ ID NO: 8 - DNA, 49 bp, Artificial Sequence
  Primer Sequence
cgctggcggcgtgcttaacacatcgcgattactagcgactccgccttca

(19) United States
(12) Patent Application Publication
(10) Pub. No.: US 2015/0126463 A1
(43) Pub. Date: May 7, 2015

(54) USE OF HERBAL SAPONINS TO REGULATE GUT MICROFLORA

(71) Applicant: Hong Kong Baptist University, Kowloon (HK)

(72) Inventors: Wen Luan Wendy Hsiao, Kowloon (HK); Lei Chen, Kowloon (HK)

(21) Appl. No.: 14/290,148

(22) Filed: May 29, 2014

Related U.S. Application Data
(60) Provisional application No. 61/899,507, filed on Nov. 4, 2013.

(51) Int. Cl. A61K 36/424; A61K 36/258; A61K 31/7034

(52) U.S. Cl. CPC A61K 36/424; A61K 31/7034; A61K 36/258
USPC 514/26

(57) ABSTRACT

The present invention relates to the use of a compound comprising plant based saponins for regulating and balancing gut microflora in a subject. The present invention also relates to the use of a compound comprising plant based saponins for exerting anti-cancer effects by regulating and balancing the gut microbial ecosystem for a subject.

### FIELD OF INVENTION

The present invention relates to the use of a plant based compound in the regulation of gut microflora in a host animal. The present invention also relates to the use of a plant based compound to exert anti-cancer effects by regulating and balancing the gut microbial ecosystem of a host animal.

### SEQUENCE LISTING

The information recorded in computer readable form is identical to the written sequence listing. With respect to ERIC1R, the primer sequence may be described as SEQ ID NO:1. With respect to ERIC2, the primer sequence may be described as SEQ ID NO:2. With respect to Bacteroides, the primer sequence may be described as SEQ ID NO: 3. With respect to Eubacteria, the primer sequence may be described as SEQ ID NO: 4. With respect to Clostridium, the primer sequence may be described as SEQ ID NO: 5. With respect to Enterococcus, the primer sequence may be described as SEQ ID NO:6. With respect to Lactobacillus, the primer sequence may be described as SEQ ID NO:7. With respect to Bifidobacterium, the primer sequence may be described as SEQ ID NO:8.

### BACKGROUND OF THE INVENTION

Normal gut microbes make significant contributions to the overall health of their host including protection against harmful microorganisms and stimulation of the immune system. Their importance can be traced back to 400 B.C., as the ancient Greek physician Hippocrates wrote, “death sits in the bowels” and “bad digestion is the root of all evil”. The intestinal tract is the primary site of interaction between the host immune system and the microbial ecosystem. The microbiome contains at least 100-fold more genes than the complete human genome, and the composition of gut microflora can likely be altered due to the plasticity of the microbiome. In healthy individuals, alterations in the microbiome composition have been linked to dietary patterns, ageing, environment and host genotype, etc. Besides the genomic influence, the host's dietary and drug uptake can also alter the composition of microflora. The fat or carbohydrate-restricted low calorie diet made obese people lose weight and result in an increase in Bacteroidetes. Conversely, the microbes can influence the energy harvesting from diet. Furthermore, the gut microbiota can also affect the bioavailability and bioactivity of ingested products, including functional foods and traditional Chinese medicine (TCM). Recent findings have revealed that the gut microflora play an even greater role in modulating human metabolic phenotypes and individuals' drug responses than previously believed. For example in Ley R E, Turnbaugh P J, Klein S, Gordon J I (2006). Microbial ecology: human gut microbes associated with obesity. Nature 444: 1022-1023 and Nicholson J K, Holmes E, Wilson I D (2005). Gut microorganisms, mammalian metabolism and personalized health care. Nat Rev Microbiol 3: 431-438, the host's dietary and drug uptake can alter the microbial composition. Conversely, microbes can influence the bioavailability and bioactivity of ingested products, including functional foods and herbal medicines. For example, recent findings in Ley R E, Turnbaugh P J, Klein S, Gordon J I (2006). Microbial ecology: human gut microbes associated with obesity. Nature 444: 1022-1023 and Ley R E, Turnbaugh P J, Klein S, Gordon J I (2006). Microbial ecology: human gut microbes associated with obesity. Nature 444: 1022-1023 indicated that the composition of two predominant gut bacterial phylum, Firmicutes and Bacteroidetes, show tight association with obesity of human and mice.

Another recent report, namely, Holmes E, Loo R L, Stamler J, Bictash M, Yap I K, Chan Q et al., (2008). Human metabolic phenotype diversity and its association with diet and blood pressure. Nature 453: 396-400, on the metabolic phenotyping of urine specimens of 4,630 participants from China, Japan, UK and USA indicated that gut microbial activities contribute to the ethnic diversity and its association with diet and blood pressure. Studies also showed that gut microbiota can alter bioavailability of intake natural products. For example, in Akao T, Kawabata K, Yanagisawa E, Ishihara K, Mizuhara Y, Wakui Y et al., (2000). Baicalin, the predominant flavone glucuronide of scutellariae radix, is absorbed from the rat gastrointestinal tract as the aglycone and restored to its original form. The Journal of pharmacy and pharmacology 52: 1563-1568, the case of the flavones baicalin isolated from scutellariae radix, the ingested baicalin is first hydrolyzed by the gut microbacteria to form the aglycone, followed by absorption and subsequently conjugated back to baicalin. Another example in Wang Y, Tang H, Nicholson J K, Hylands P J, Sampson J, Holmes E (2005). A metabonomic strategy for the detection of the metabolic effects of chamomile (Matricaria recutita L.) ingestion. Journal of agricultural and food chemistry 53: 191-196, showed that functional food chamomile tea altered the metabolites and bacterial composition.

Metabolic activation of ginseng saponins, ginsenosides by intestinal bacteria have also been investigated extensively. Other natural products isolated from TCM have also been proven to be metabolized by gut microbes to form active drugs, such as glycyrrhizin, paeoniflorin, baicalin, puerarin and daidzin. Nevertheless, studies have been confined to the metabolites of the TCM, and no systematic study of the alteration of the microflora under the influence of ingested herbal medicines.

It is possible that TCM with a longer residence time in the intestinal tract may have a great chance to affect the gut microbial ecosystem. Saponins are the natural triterpenoids found in many herbal and edible plants. Saponins have the following traits underlying poor membrane permeability and result in poor intestinal absorption, relative high molecular mass (>500 Da), high hydrogen-bonding capacity (>12) and high molecular flexibility (>10). These non-absorbable saponins are too difficult to be absorbed through the intestinal wall and able to interact with gut microflora for a longer time. Saponins are commonly found in a large number of natural sources and particularly abundant in many herbal and edible plants. They are a group of amphiphilic glycosides containing one or more sugar chains bound to a nonpolar triterpene (FIG. 1A) or steroid aglycone (FIG. 1B) skeleton.

Citation or identification of any reference in this section or any other section of this application shall not be construed as an admission that such reference is available as prior art for the present application.

### SUMMARY OF INVENTION

In accordance with a first aspect of the present invention, there is provided a use of a composition comprising saponins extracted from plants for improving gut microbial ecosystem of a subject.

In an embodiment of the first aspect, the plants comprising Gynostemma pentaphyllum (Gp), Panax pseudoginseng, Panax notoginseng and Panax ginseng.

In an embodiment of the first aspect, the Panax ginseng is processed to comprise red ginseng.

In an embodiment of the first aspect, the Panax ginseng is processed by steaming.

In an embodiment of the first aspect, the plants further comprising Radix Notoginseng of Panax pseudoginseng, Radix Notoginseng of Panax notoginseng and Radix Ginseng of Panax ginseng.

In an embodiment of the first aspect, the saponins are of a range of concentration of about 500 mg/kg to 750 mg/kg in the composition.

In an embodiment of the first aspect, the improvement to the gut microbial ecosystem comprising regulating and balancing the gut microbial ecosystem by increasing symbionts in the gut ecosystem of said subject.

In an embodiment of the first aspect, said subject is a human.

In an embodiment of the first aspect, the composition is used as prebiotics for improving the gut microbial ecosystem of a subject.

In an embodiment of the first aspect, the improvement of the gut microbial ecosystem of a subject results in an inhibitory effect on tumor growth in said subject.

Other aspects and advantages of the invention will become apparent to those skilled in the art from the following description of the drawings, which are given by way of example only to illustrate the invention.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 shows the chemical structures of common saponins including (FIG. 1A) triterpenoid saponins and (FIG. 1B) steroid saponins.

FIG. 2A shows a schematic diagram illustrating the experimental design;

FIG. 2B shows the ERIC-PCR fingerprints of the control group;

FIG. 2C shows the ERIC-PCR fingerprints of the Gp saponins (GpS) treatment group;

FIG. 2D shows the ERIC-PCR fingerprints of the Notoginseng saponins (NGS) treatment group;

FIG. 2E shows the ERIC-PCR fingerprints of the red ginseng saponins (RGS) treatment group;

FIG. 2F shows the ERIC-PCR fingerprints of the ginseng saponins (GS) treatment group;

FIG. 2G-2I show the ERIC-PCR data analysis of different treatment groups:

FIG. 2G shows the band numbers of the different treatment groups;

FIG. 2H shows the Shannon-Wiener diversity index (H′ index) of the different treatment groups;

FIG. 2I shows the Sorenson's pairwise similarity coefficient (Cs) of the different treatment groups;

FIG. 3A shows the results of control versus Gp saponins treated mice;

FIG. 3B shows the results of control versus notoginseng saponins treated mice;

FIG. 3C shows the results of control versus red ginseng saponins treated mice;

FIG. 3D shows the results of control versus ginseng saponins treated mice.

FIG. 4A shows the intensity of the 1200 bp fragment among different treatment group;

FIG. 4B shows the intensity of the 950 bp fragment among different treatment group;

FIG. 4C shows the intensity of the 230 bp fragment among different treatment group;

FIG. 4D shows that intensity of the 210 bp fragment among different treatment group.

FIG. 5A shows the effect of different plant based saponins including Gp, notoginseng, red ginseng and ginseng saponins and control on Bacteroides as determined by 16S rRNA PCR.

FIG. 5B shows the effect of different plant based saponins including Gp, notoginseng, red ginseng and ginseng saponins and control on Bifidobacterium as determined by 16S rRNA PCR.

FIG. 5C shows the effect of different plant based saponins including Gp, notoginseng, red ginseng and ginseng saponins and control on Clostridium as determined by 16S rRNA PCR.

FIG. 5D shows the effect of different plant based saponins including Gp, notoginseng, red ginseng and ginseng saponins and control on Enterococcus as determined by 16S rRNA PCR.

FIG. 5E shows the effect of different plant based saponins including Gp, notoginseng, red ginseng and ginseng saponins and control on Lactobacillus as determined by 16S rRNA PCR.

FIG. 6A-6E show the 3D score plot of principal component analysis (PCA) of MS data showing the comparison of fecal metabolic profiles among different treatment groups in ESI positive mode.

FIG. 6F shows the representative base peak chromatogram of the fecal metabolites;

FIG. 6G shows the results of heatmap analysis of discriminative metabolites in fecal samples from control mice, red ginseng and ginseng saponins treated mice.

FIG. 7A shows the representative ERIC-PCR fingerprints of the fecal microflora of individual normal and xenograft nude mice. Fecal samples were collected before xenograft (Day 0), and 5 & 10 days upon saline or tumor cells injection; A1-3: three control mice; B1-3: three xenograft nude mice.

FIGS. 7B and 7C show the digitization of ERIC-PCR fingerprints.

FIG. 7D shows the PLS-DA plot of ERIC-PCR data from fecal microflora of normal and xenograft nude mice at Day 10. Box: the normal nude mice; Dot: the xenograft nude mice;

FIG. 7E shows the correlation coefficients of fecal microflora of normal and xenograft nude mice.

FIG. 8A shows the effect of GpS on tumor growth in nude mice showing the tumor volume;

FIG. 8B shows the effect of GpS on tumor growth in nude mice showing the tumor weight;

FIG. 8C shows the effect of GpS on tumor growth in nude mice showing the body weight;

FIG. 9A shows a schematic diagram of experimental design for the effect of GpS on the composition of fecal microflora in normal nude mice;

FIG. 9B shows the PLS-DA score plots of ERIC-PCR data of the control group in normal nude mice (n=6);

FIG. 9C shows the PLS-DA score plots of ERIC-PCR data of the GpS treatment group in normal nude mice (n=6);

FIG. 9D shows a schematic diagram of experimental design for the effect of GpS on the composition of fecal microflora in xenograft nude mice;

FIG. 9E shows a PLS-DA score plots of ERIC-PCR data of the control group in xenograft nude mice (n=7);

FIG. 9F shows a PLS-DA score plots of ERIC-PCR data of the GpS treatment group in xenograft nude mice (n=7);

FIG. 9G shows a schematic diagram of experimental design for comparing composition of fecal microflora between the control and GpS groups in xenograft nude mice with antibiotic intervention;

FIG. 9H shows the PLS-DA score plots of ERIC-PCR data of the control group in xenograft nude mice with antibiotic intervention (n=3);

FIG. 9I shows the PLS-DA score plots of ERIC-PCR data of the GpS treatment group in xenograft nude mice with antibiotic intervention (n=3).

FIG. 10A shows the OUT network of fecal samples from normal and xenograft nude mice;

FIG. 10B shows the numbers of shared and unique OTUs of normal and xenograft nude mice;

FIG. 10C shows the diversity of fecal microflora in normal and xenograft nude mice;

FIG. 10D shows the taxonomic representations of fecal microbiome of normal and xenograft nude mice;

FIG. 10E shows the histogram of the LDA scores of fecal 16S rRNA sequences of normal (white) and xenograft (black) mice;

FIG. 10F shows the relative abundance of differentially abundant families and genera between normal and xenograft mice;

FIG. 11A shows a bar chart of relative abundance of bacterial phyla in nude mice with or without GpS treatment;

FIG. 11B shows changes in relative abundance of the main phyla of microbial communities in the gut.

FIG. 12A shows the taxonomic representations of fecal microbiome of normal nude mice with or without GpS treatment;

FIG. 12B shows the histogram of the LDA scores for differentially abundant clades. White: samples from controls; Black: samples from normal nude mice with 10 days of GpS treatment;

FIG. 12C shows the relative abundance of differentially abundant families and genera in normal nude mice with or without GpS treatment (Control group, n=3; GpS group, n=3);

FIG. 12D shows the taxonomic representations of fecal microbiome of xenograft nude mice with or without GpS treatment;

FIG. 12E shows the histogram of the LDA scores for differentially abundant taxa. White: samples from controls; Black: samples from xenograft nude mice with 10 days of GpS treatment;

FIG. 12F shows the relative abundance of differentially abundant families and genera in xenograft nude mice with or without GpS treatment (Control group, n=3; GpS group, n=3).

FIG. 13A shows the Venn diagram showing the number of unique and shared bacterial families between normal and xenograft nude mice with or without GpS treatment;

FIG. 13B shows the unique families between normal and xenograft nude mice; normal nude mice with or without GpS treatment; xenograft nude mice with or without GpS treatment (n=3 per group).

FIG. 14A shows the OTU heatmap of identified bacterial species;

FIG. 14B shows the corresponding OTU IDs of FIG. 14A with the respective identified bacterial species;

FIG. 14C shows the relative abundance of Clostridium cocleatum in normal and xenograft nude mice with or without GpS treatment. Data are presented as mean±SEM (n=3 per group);

FIG. 14D shows the relative abundance of Bacteroides acidifaciens in normal and xenograft nude mice with or without GpS treatment. Data are presented as mean±SEM (n=3 per group).

FIG. 15 shows the quality control of GpS.

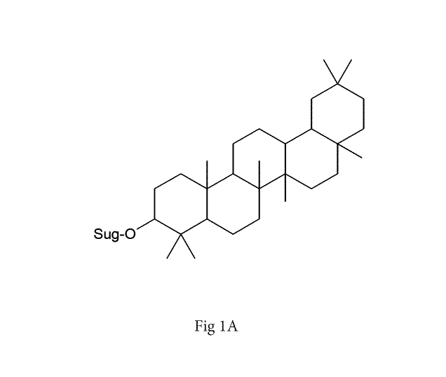

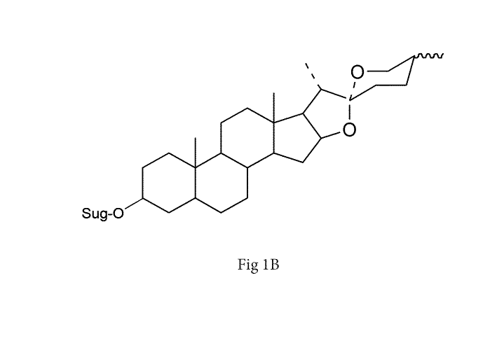

### DETAILED DESCRIPTION OF THE PREFERRED EMBODIMENTS

The present invention is not to be limited in scope by any of the specific embodiments described herein. The following embodiments are presented for exemplification only.

The present invention relates to a composition comprising saponins extracted from plants for improving gut ecosystem of a subject. In particular, the present invention relates to the use of saponins from Gynostemma pentaphyllum (Gp), Radix Notoginseng of Panax pseudoginseng (or Panax notoginseng), Radix Ginseng of Panax ginseng and red ginseng (steamed Panax ginseng) in regulating and balancing the gut microbial ecosystem by increasing symbionts. The present invention also has possible application in treatment with saponins from Gynostemma pentaphyllum (Gp) to exert anti-cancer effects by regulating and balancing the gut microbial ecosystem for the host animal.

Saponins from Gynostemma pentaphyllum (Gp), Radix Notoginseng, Radix Ginseng and Red Ginseng (Steamed Panax ginseng) and the Effects on Gut Microflora

Saponins from four famous plant based or herbal Chinese medicines are involved in this example of the present invention, including Gynostemma pentaphyllum (Gp), Radix Notoginseng of Panax pseudoginseng (or Panax notoginseng), Radix Ginseng of Panax ginseng and red ginseng (steamed Panax ginseng). Triterpenoid saponins are the major compounds in these herbal medicines and are considered to be the main bioactive components responsible for a variety of pharmacological activities.

Materials and Methods

The chemical figure printings of the four plant based saponins (from Gynostemma pentaphyllum (Gp), Radix Notoginseng, Radix ginseng and red ginseng (steamed Panax ginseng)) were performed according to Wu P K, Tai C S, Choi C Y, Tsim W K, Zhou H, Liu X et al., (2011) Chemical and DNA authentication of taste variants of Gynostemma pentaphyllum herbal tea. Food Chemistry 128: 70-80.

Animals and Treatments

Animal welfare and experimental procedures were performed strictly in accordance with the care and use of laboratory animals. All procedures were approved by the University Ethics Review Committee for animal research. The C57BL/6 mice (8 weeks old) were purchased from Chinese University of Hong Kong, on a 12-h light/dark cycle and with free access to food and water. Total saponins of Gynostemma pentaphyllum (GpS), Radix Notoginseng (NGS), Radix ginseng (GS) and red ginseng (RGS) were dissolved in milli-Q H₂O at 50 mg/ml respectively and then filtered (0.2 μm). Single dose of these four different saponins at 500 mg/kg or milli-Q H₂O control was given to different treatment groups of mice daily by gavage, started the second day after the first fecal samples collection. For experimental animal, fecal samples were collected (8:00-10:00 a.m.) at day 0 (before treatment), and 5 days, 10 days and 15 days after treatment. All fecal samples were immediately stored at −20° C. and kept for later DNA extraction.

Bacterial Genomic DNA Extraction from Fecal Samples

Total genomic DNA was isolated from fecal samples as described with slight modification as in the previous study. 0.1 g of fecal samples were vortexed in 4 ml sterile PBS (pH 7.4) for 5 minutes, then centrifuged at 40×g for 8 minutes to collect the upper phase containing the bacteria. After repeating this procedure once, the supernatant was centrifuged at 2000×g for 8 minutes. The supernatant was discarded and the bacterial pellets were then washed twice with PBS. The bacterial pellets were used for DNA extraction as described. The DNA concentration was determined by NanoDrop 1000 spectrophotometry.

ERIC (Enterobacterial Repetitive Intergenic Consensus)-PCR

ERIC sequences are non-coding, highly conserved intergenic repeated sequences that reside in the genome of various bacterial species in addition to enterobacteria as it was first discovered. ERIC-PCR was used to profile the gut microbiome using fecal genomic DNA as the template and a pair of ERIC specific primer sequences: ERIC 1R (5′-ATGTAAGCTCCTGGGGATTCAC-3′) and ERIC 2 (5′-AAGTAAGTGACTGGGGTGAGCG-3′). A 25 μl reaction mixture containing 5 μl 5×PCR reaction buffer, 200 μM dNTP, 2.5 mM Mg²⁺, 0.4 μM primers, 1 unit Hotstart Taq polymerase, and 50 ng fecal genomic DNA. PCR was performed under the following conditions: an initial denaturation at 94° C. for 5 minutes, followed by 35 cycles of denaturing at 95° C. for 50 seconds, annealing at 49° C. for 30 seconds, 46° C. for 30 seconds, and extension at 72° C. for 3 minutes; and then a final extension at 72° C. for 9 minutes. 10 μl of each PCR product was loaded into a 2% (w/v) agarose gel containing 0.5 μg/ml ethidium bromide and run for 40 minutes at 100 V in 1×TAE buffer. A DNA ladder (0.1-10.0 kb) was used as DNA marker (NEB, N3200). Agarose gels were photographed using a Gel Doc™ XR+ System.

Data Analysis of ERIC-PCR Fingerprints

Partial least squares discriminant analysis (PLS-DA) was performed to visualize the dynamic changes of microflora composition before and after treatment. Based on the distance and the intensity of each DNA bands (lane %), the banding patterns of ERIC-PCR products separated on the gel were digitized by Image Lab 3.0 system (Bio-Rad) and performed PLS-DA analysis using SIMCA-P 12.0 tool. Sorenson's pairwise similarity coefficient (Cs) was used to perform a paired comparison on the microflora profiles before and after treatment. Two identical profiles create a Cs value of 100%, whereas two completely different profiles (no common bands) result in a Cs value of 0%. Cs (%)=(2×j)/(a+b)×100%, where a is the number of total bands in the ERIC-PCR pattern for one sample, b is the number for the other, and j is the number of the common bands shared by the two samples. Shannon-Weiner diversity index, also called H′ index, refers to the community richness, was used to describe the microflora distribution of PCR bands in our study, although each ERIC-PCR band does not have to stand for one individual bacterial species. H′=Σ−(Pi*ln Pi), where pi refers to the relative abundance of each band in the lane of the fingerprint (lane %).

Identification of Bacterial Species Using 16S rRNAPCR

16S rRNA PCR was used to detect major bacteria genera. Primers specific to 16S rRNA of all eubacteria were used as an endogenous control to normalize gene intensity data between different samples. All primer sets used are listed in Table 1. Each PCR mixture (25 μl) contained 5 μl 5×PCR reaction buffer, 200 μM dNTP, 2.5 mM Mg²⁺, 0.4 μM primers, 1 unit Taq polymerase, and 50 ng fecal genomic DNA. The optimal annealing temperature for each primer set was determined by using a gradient PCR program (Applied Biosystems Veriti™ Thermal Cycler). The amplification conditions were one cycle at 95° C. for 5 minutes followed by the indicated cycles (see Table 1) at 95° C. for 30 seconds, the indicated annealing temperature (see Table 1) for 1 minute, 72° C. for 1 minute, final extension at 72° C. for 8 minutes and then cooling to 4° C. PCR products were examined for expected bands on 1% or 2% (according to the size of PCR product) agarose gel containing 0.5 μg/ml ethidium bromide by running 10 μl of the PCR product. The size of the PCR fragments was determined using a 1 kb DNA ladder. The agarose gels were photographed using a Gel Doc™ XR+ System and digitized by Image Lab 3.0 system (Bio-Rad).

TABLE 1
16S rRNA PCR Primer Used in This Study

| | | Product | Annealing | |
|---|---|---|---|---|
| Bacteria | Primer Sequence (5′ to 3′) | Size (bp) | Temp (° C.) | Cycles |
| All eubacteria | TCCTACGGGAGGCAGCAGT | 466 | 62 | 22 |
| | GGACTACCAGGGTATCTATCCTGTT | | | |
| Bacteroides | GGGGTTCTGAGAGGAAG | 950 | 52 | 25 |
| | ACCCCCCATTGTACCAC | | | |
| Clostridium. | AAAGGAAGATTAATACCGCATAA | 722 | 54 | 28 |
| | ATCTTGCGACCGTACTCCCC | | | |
| Enterococcus | CCCTTATTGTTAGTTGCCATCATT | 144 | 56 | 30 |
| | ACTCGTTGTACTTCCCATTGT | | | |
| Lactobacillus | GGAATCTTCCACAATGGACG | 216 | 61 | 30 |
| | CGCTTTACGCCCAATAAATCCGG | | | |
| Bifidobacterium | CGCTGGCGGCGTGCTTAACACAT | 1300 | 60 | 22 |
| | CGCGATTACTAGCGACTCCGCCTTCA | | | |

Metabonomic Study

A metabolomic study on the fecal samples collected from different plant based saponins treated mice was performed by using ultra high-performance liquid chromatography (UHPLC) coupled with quadrupole time-of-flight (Q-TOF) mass spectrometry. The Mass Profiler Professional (MPP) B.02.00 software was used to analyze the metabolomic data. The metabolites of fecal samples were extracted with methanol. The volume of 100% methanol in the extraction was 250 μl per 0.1 g of feces. Fecal samples were homogenized in methanol, followed by vortexing and incubating for 15 min at room temperature and then centrifuged at maximum speed (˜20000 g) for 15 min. The supernatant was transferred and filtered (0.22 um Hydrophilic PVDF, Millipore). The metabolite extracts were frozen at −20° C. until analysis. The chromatography was performed on Agilent 1290 Infinity UHPLC equipped with G4220A binary pump, G4226A automatic sample injector and G4212A Diode Array Detector (Agilent Technologies, Santa Clara, Calif., USA). The separation was conducted with an ACQUITY UPLC BEH C8 column, 2.1×100 mm i d, 1.7 μm (Waters Corp., Milford, Mass., USA). A mobile phase consisted of 0.1% acetic acid and 5 mM ammonium acetate in milli-Q water (A) and acetonitrile (B) was used for separation. The system was programmed with the following gradients: 0-0.25 min, 10% B; 0.25-5 min, 10-75% B; 5-22 min, 75-99% B; 22-27 min, 99% B. The flow rate was kept constant at 0.4 ml/min at 45° C. for a total run time of 30 min. The volume of sample injection was 8 μl. An Agilent 6540 Ultra High Definition (UHD) Accurate-Mass Q-TOF mass spectrometer (Agilent Technologies, Santa Clara, Calif., USA) was coupled to the UHPLC system described above via an electrospray ionization (ESI) ion source with Jet-Stream technology for the comprehensive LC/MS analysis of fecal samples. The ESI-MS spectra were acquired in the positive and negative ion modes. Ultra-high-purity nitrogen was used as collision gas in product ion scanning experiments. The capillary voltage was set at 4.5 kV. The drying gas and sheath gas were delivered at flow rate of 8 L/min and temperatures were 300° C. and 350° C., respectively. The pressure of nebulizer gas was 35 psi. The fragmentor voltage is 135 V. The mass analyzer was scanning from 80 to 1700 (m/z). Data were collected at a spectral acquisition rate of 2 Hz. MassHunter Qualitative Analysis was used to create the Molecular feature extraction (MFE) method for the metabolomics data. MassHunter DA Reprocessor was then used to automate MFE on all of the samples in a single batch processing. The molecular features for each sample data file were exported as a CEF file and imported into MPP software. Principal Component Analysis (PCA) was used to find differences between samples and weigh relative contributions of compounds to the separation of the groups by MPP. A series of differential metabolites were obtained and heatmap was generated based on statistical analysis (Oneway ANOVA, p<0.05) by MPP.

Statistical Analysis

The data obtained are presented as means±SEM., and statistical comparisons were performed using one-way ANOVA followed by Student's t-test at P values of <0.01(**) or <0.05(*).

Results

Chemical Profiles of Four Plant Based Saponins

ERIC-PCR Fingerprint of Fecal Microflora in Plant Based Saponins Treated Mice

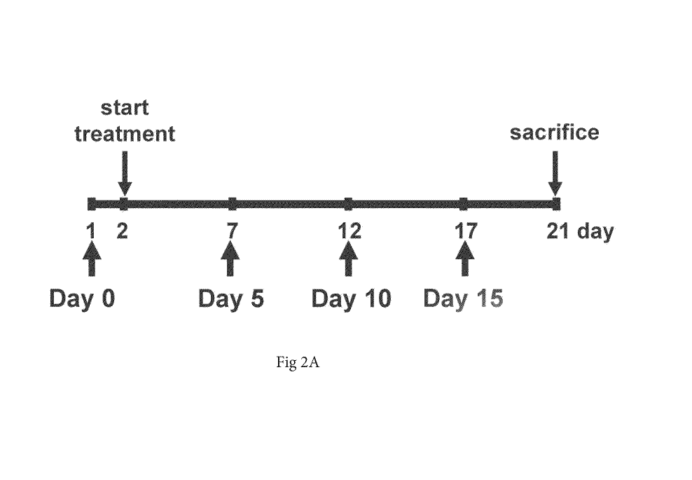

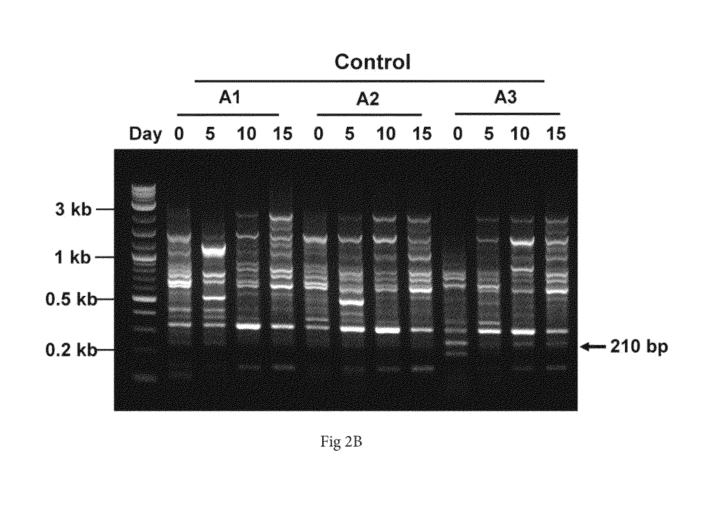

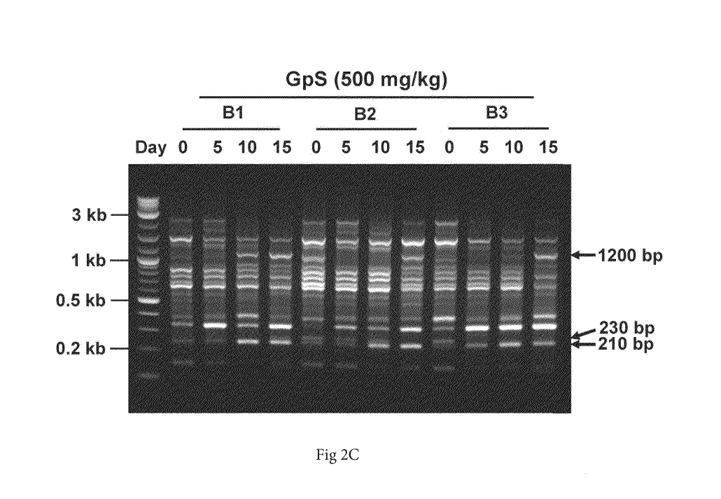

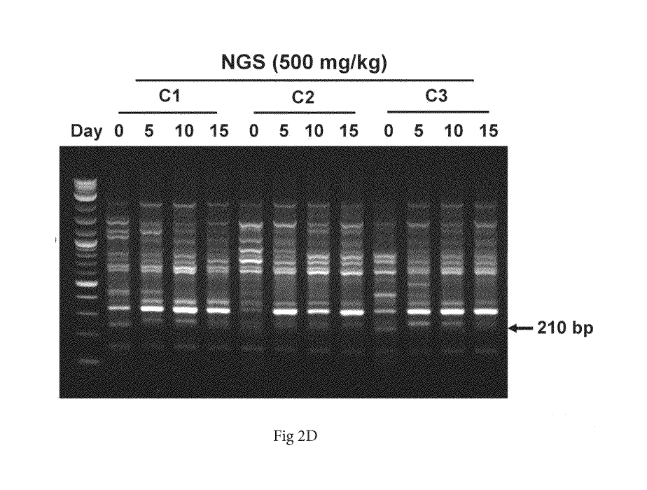

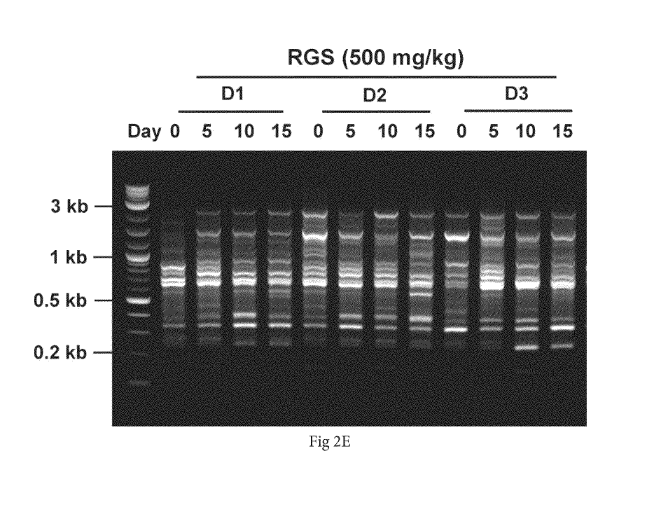

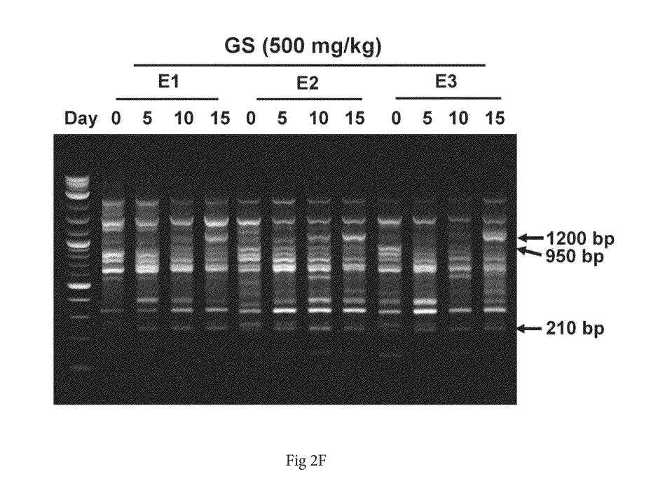

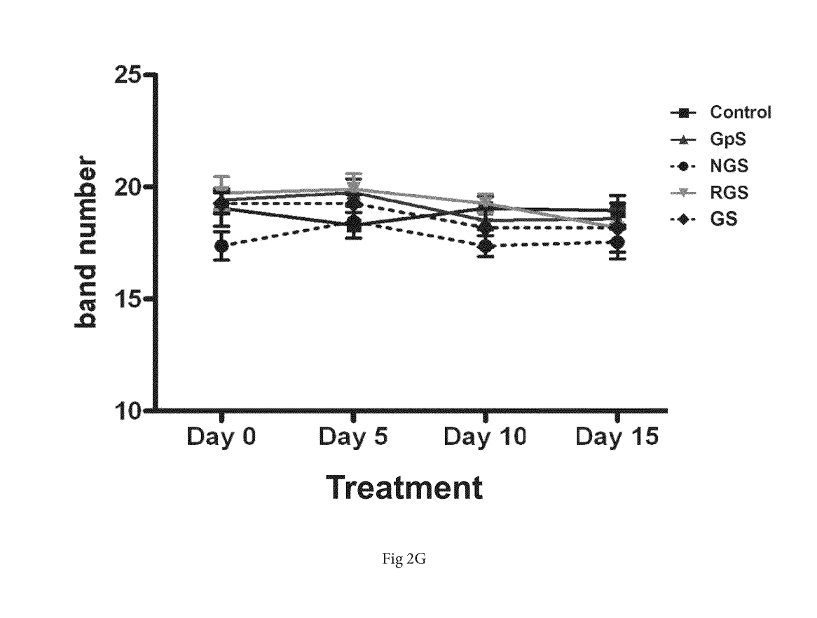

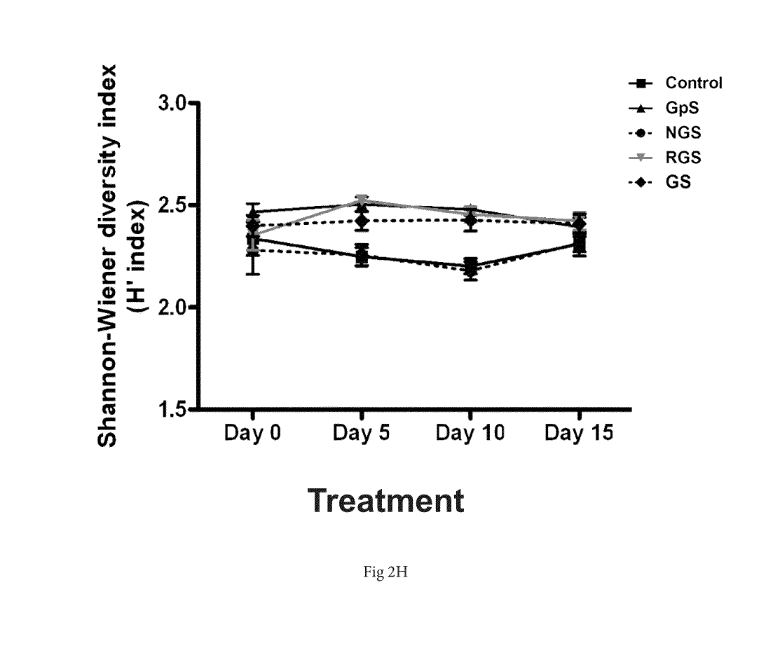

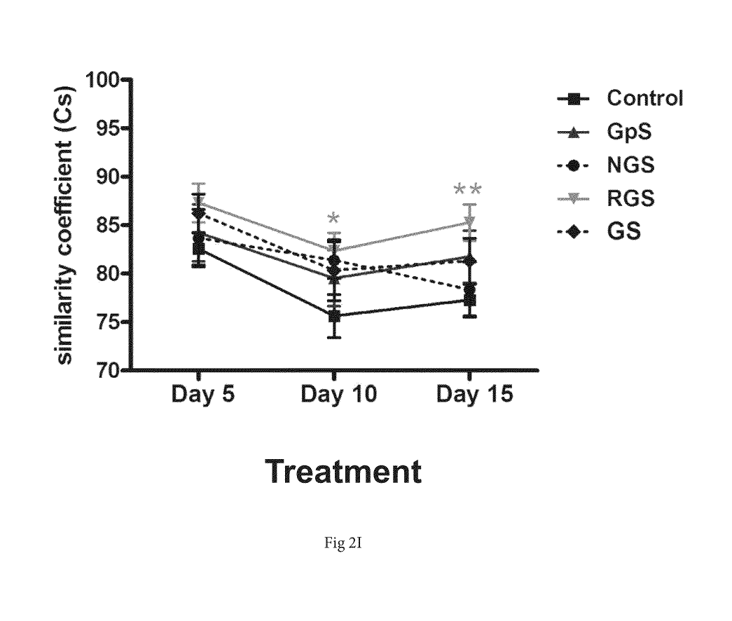

To investigate how plant based saponins would affect the gut microflora composition in the normal mice, fecal samples were collected from the five groups including control group and four different plant based saponins treatment groups at Day 0, Day 5, Day 10 and Day 15 as described (see FIG. 2A-2F). Genomic DNA isolated from the fecal samples was analyzed by ERIC-PCR. Among all the treated mice, the fecal microbial fingerprints showed an average of 19 fragments per sample, ranging from approximately 100 to 3000 bp with various intensities (FIG. 2A-2F). There were no significant differences in the numbers of ERIC-PCR fragments among different treatment groups (FIG. 2G) as well as the Shannon-Wiener diversity index (FIG. 2H). On the other hand, the similarities between samples were evaluated by calculating Sorenson's pairwise similarity coefficient (Cs). For individual mice, the microbial profiles of Day 5, Day 10 and Day 15 samples were compared to their Day 0 status (before treatment). Two identical profiles and two completely different profiles create Cs value of 100% and 0%, respectively. Compared to the control group, the samples collected on different days from the same mouse showed a better consistency in plant based saponins treated groups. The fecal microbial composition showed a higher Cs ranging from 76% to 97% in mice with red ginseng saponins treatment, followed by ginseng (65%-95%) and Gynostemma pentaphyllum saponins (62%-95%). However, the similarity coefficient in the mice with the treatment of notoginseng saponins was closer to the control mice at Day 15 (FIG. 2I).

Plant Based Saponins Altered the Fecal Microbial Composition

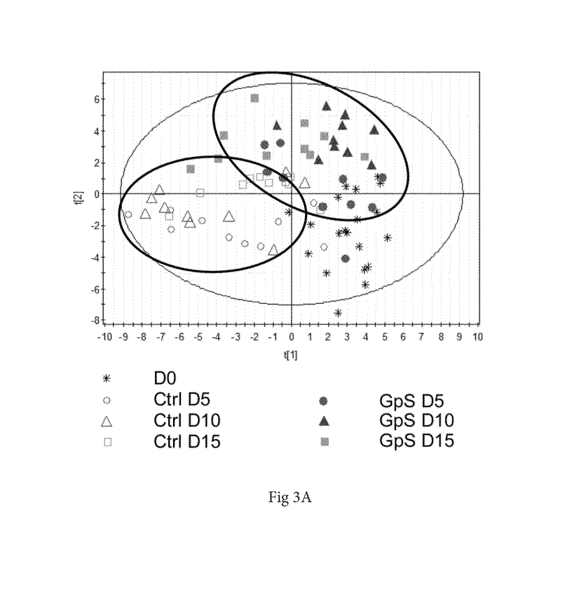

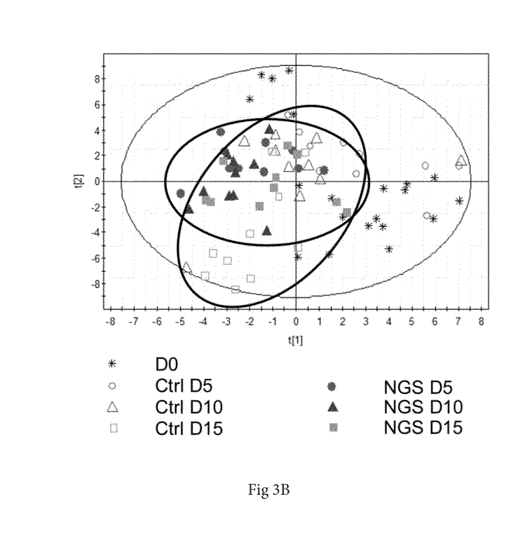

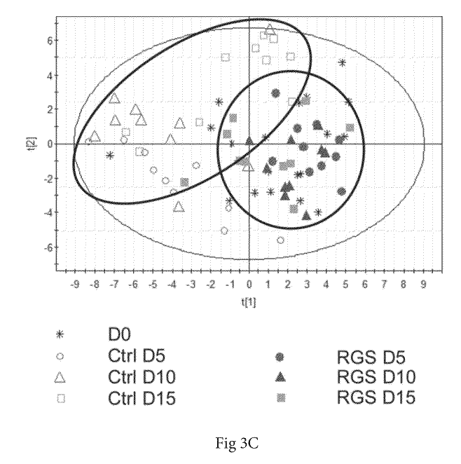

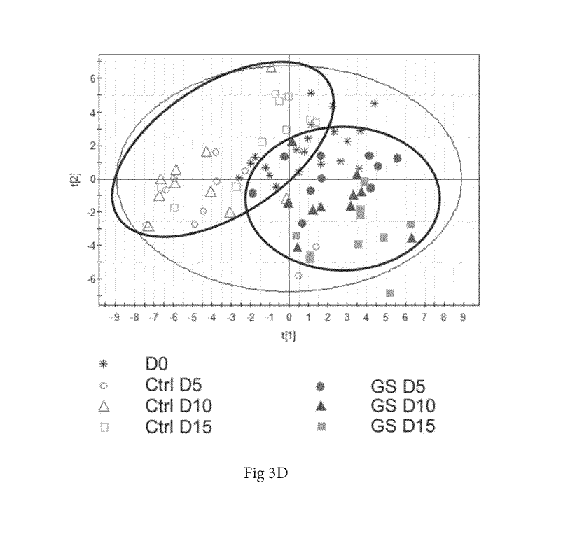

The PLS-DA plots, based on the ERIC-PCR banding patterns, displayed a clear alteration of microflora profiles in the plant based saponins treatment groups in contrast to the control group. The fecal microflora composition was fixed in a relatively stable pattern after plant based saponins treatment at different time points. The fecal microflora communities in plant based saponins treated mice clustered in an area that remained distinct from that of controls (FIG. 3A-3D). However, there was some overlapping between the samples from control and notoginseng saponins treated mice. When comparing the fecal microbial composition before and after treatment, the cluster of samples from red ginseng saponins treated mice was quite close to Day 0 status (before treatment). This finding was consistent with the highest Cs value in red ginseng saponins treatment group. Interestingly, among the four plant based saponins, red ginseng and ginseng saponins yielded relatively similar patterns of microbial composition.

Fecal Microflora Showed Differential Response to Different Plant Based Saponins

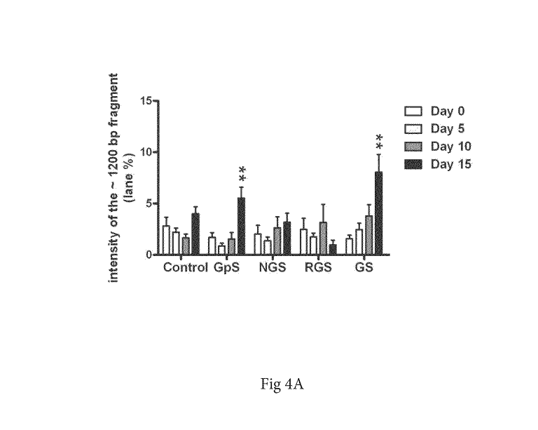

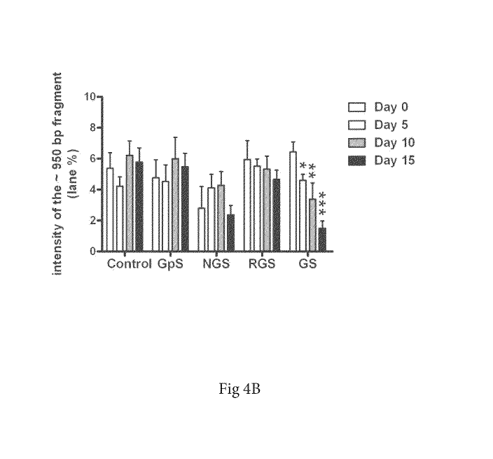

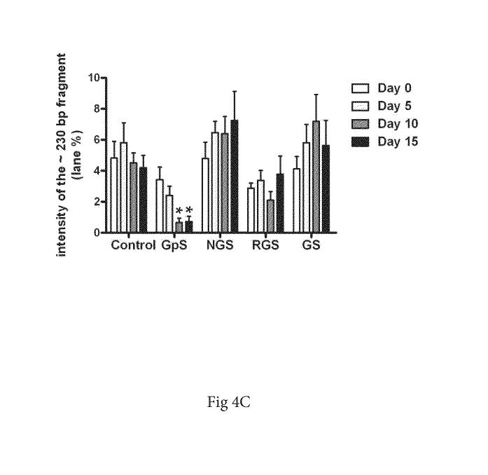

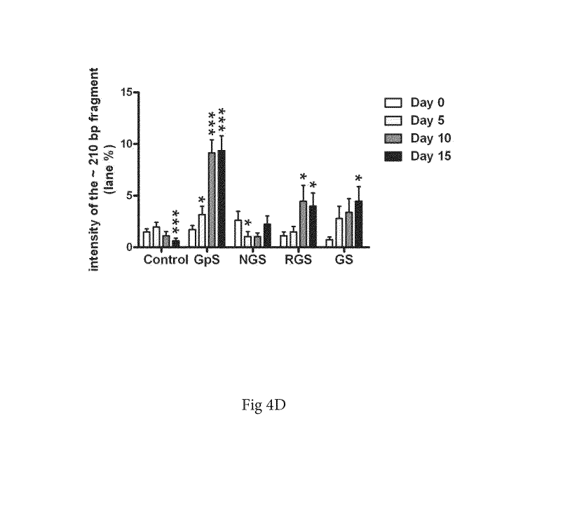

We then further identified the differential ERIC-PCR fragments between saponins treated mice and controls. As shown in FIG. 4A-4D (arrows indicated these fragments in the fingerprints in FIG. 2A-2F), Gp and ginseng saponins can increase the intensity of the 1200 bp fragment. Additionally, ginseng saponins can decrease the intensity of the 950 bp fragment, while Gp saponins can reduce the 230 bp band. Furthermore, the intensity of 210 bp fragment was significantly down-regulated in controls, whereas the three plant based saponins, Gp, red ginseng and ginseng saponins, can enhance this band after treatment. All these discriminative ERIC-PCR fragments further revealed the differential response of the gut microbiota to the treatment of different plant based saponins, and can be the main contributors to the distinct ERIC-PCR profiles among different treatment groups.

Plant Based Saponins Differentially Affect Major Bacterial Genera Detected in Mice

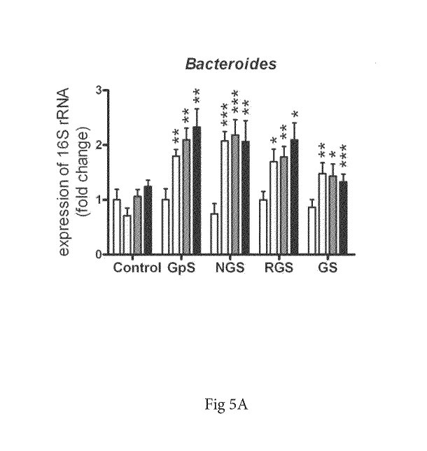

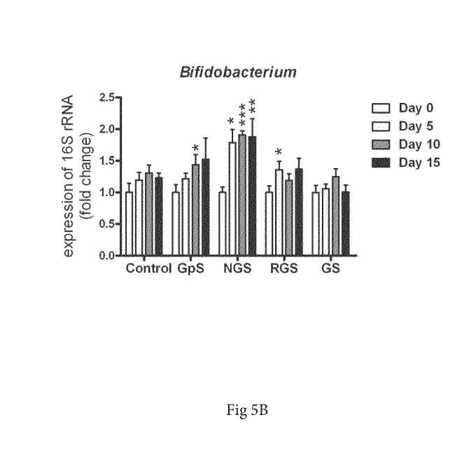

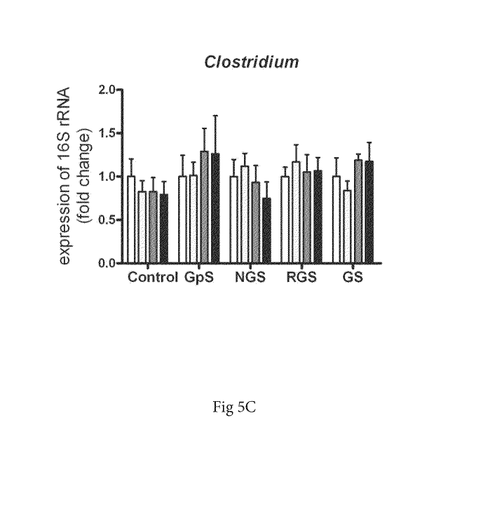

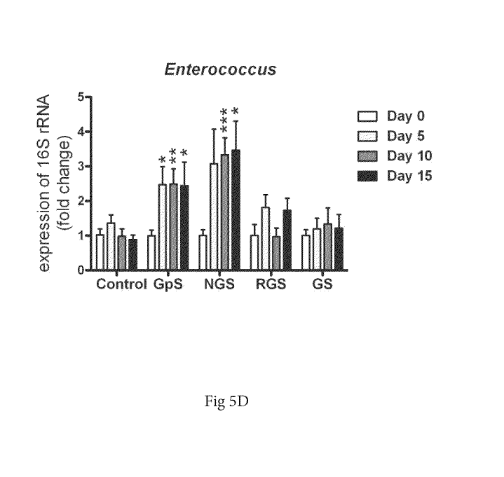

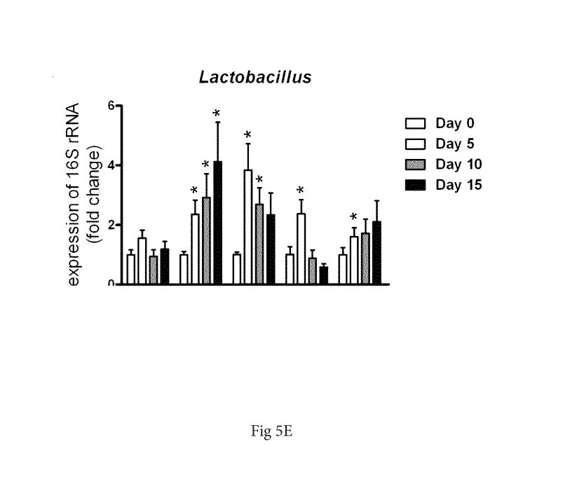

We also investigated the major bacterial genera in the feces of plant based saponins treated mice by 16S rRNA PCR. There are two dominant bacterial phyla in the gut ecosystem, Gram-positive Firmicutes (most notably Clostridium spp., Enterococcus spp. and Lactobacillus spp.) and Gram-negative Bacteroidetes (Bacteroides spp.). Using PCR primer sets of the 16S rRNA specific for the above bacteria genera as well as Bifidobacterium spp., we found that these four plant based saponins all can significantly enhance the level of Bacteroides (FIG. 5A); Gp and notoginseng saponins showed much more effect on the increase of Bifidobacterium, Lactobacillus, as well as Enterococcus, and no obvious changes in the level of Clostridium in all of the treatment groups (FIG. 5B-5E).

Plant Based Saponins Altered the Fecal Metabolites

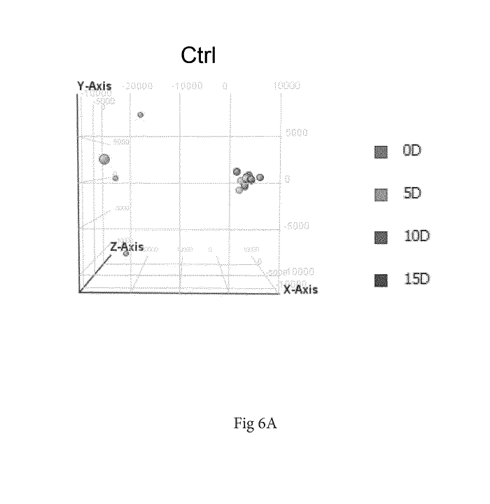

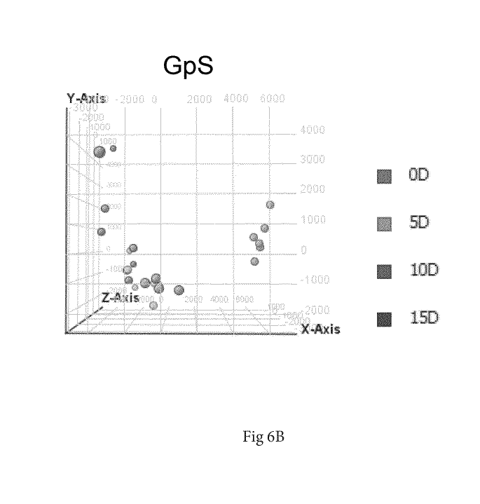

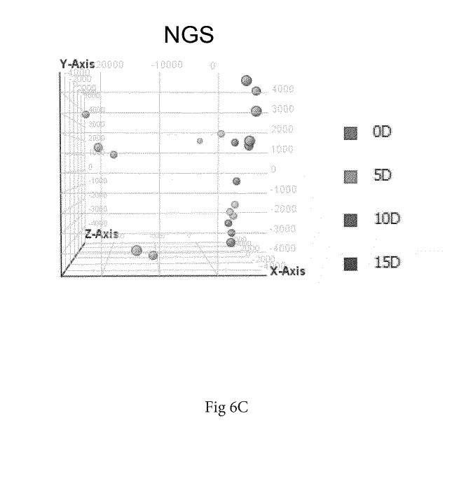

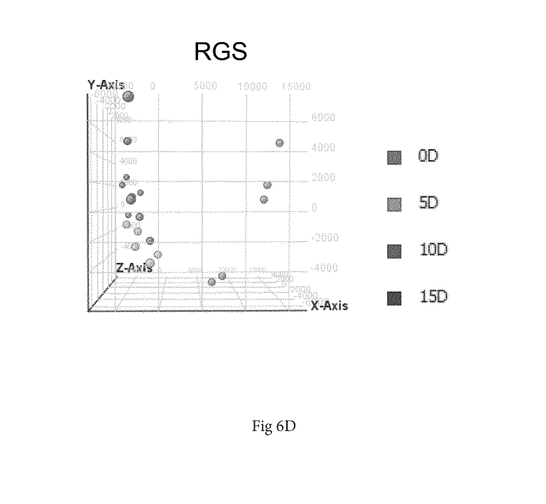

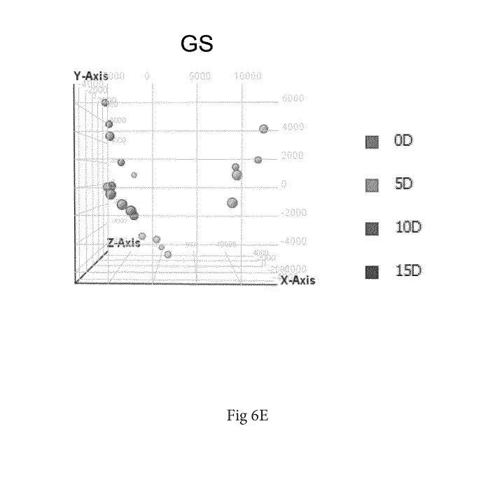

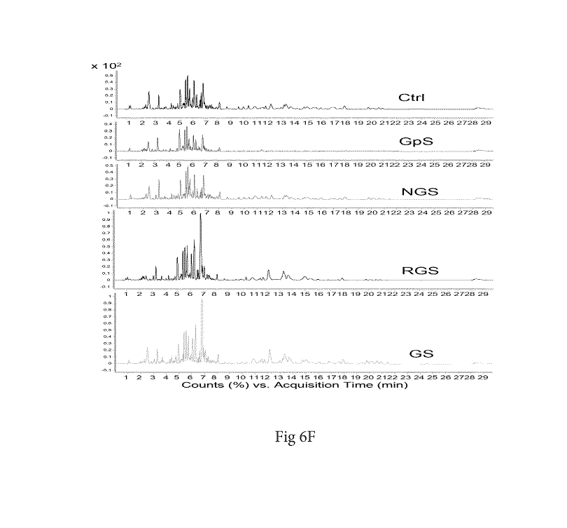

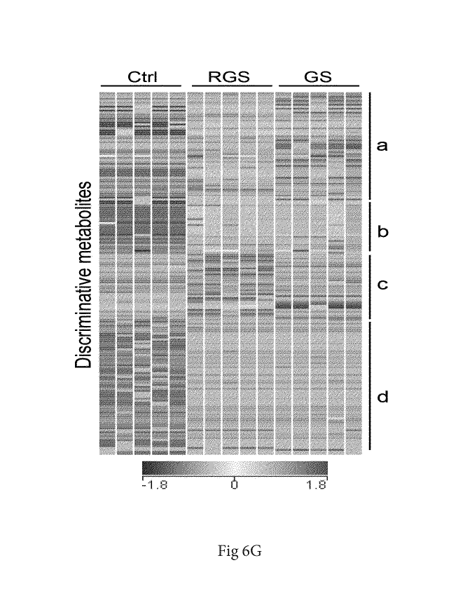

To investigate the effect of plant based saponins on the fecal metabolites, an ultrahigh-performance liquid chromatography coupled with Q-TOF mass spectrometry was performed to obtain the fecal metabolic profiles. The acquired data were subjected to principal component analysis by using MPP software. As shown in FIG. 6A-6E, fecal metabolites from control mice at Day 5, Day 10 and Day 15 clustered together. However, plant based saponins-treated mice showed a shift in a time-dependent manner. By comparing base peak chromatogram (FIG. 6F), it seemed that the fecal metabolic profile of notoginseng saponins-treated mice was closest to that of the control mice at Day 15, which was consistent with the comparative results of fecal microbiota among different treatment groups. On the other hand, we found red ginseng saponins and ginseng saponins dramatically altered the fecal metabolites. In contrast to Gp saponins and notoginseng saponins, the metabolomic profiles of red ginseng and ginseng saponins showed a relatively similar pattern although the differences can also be found within the two groups. Then we generated a heatmap (FIG. 6G) by MPP software to further reveal the general pattern of discriminative metabolites between these two groups. Compared to the control group, roughly half of the discriminative metabolites (b & d) showed similar changes in red ginseng and ginseng saponins treatment groups. Meanwhile, some metabolites showed different degrees of alteration (a) or an opposite alteration (c).

### DISCUSSION

Most current drug development is focused on identifying a novel candidate against a specific target, for example, a receptor or an enzyme. However, gut microbial ecosystem has long been underestimated. The gut microbiota is now proposed to be a potential therapeutic strategy, as well as a big treasury for drug development. Traditional Chinese Medicine is believed to modulate homeostasis by balancing Yin and Yang. It is possible that TCM can also restore the balance of the gut microbial system, thus achieving homeostasis and producing therapeutic effects in the host. However, the research on the effect of plant based medicines on gut microflora is very limited, and their mediated interactions between host and microflora have been seldom investigated.

Recent findings have already revealed that saponins can be hydrolysed by intestinal flora. After absorption, the deglycosylated metabolites (aglycones) undergo phase I and/or II metabolism. But how would gut microflora respond to the treatment of plant based saponins? We hypothesize that plant based saponins may change the composition of gut microflora, which in turn alter the metabolites through host-microbe interactions. As the first step towards understanding the role of the microflora in host drug responses, we try to understand the association between gut microflora and plant based saponins.

The chemical profiles, microbial profiles and metabolic profiles in plant based saponins treated mice have been investigated in this example. Ginseng (Panax ginseng C. A. Meyer, Araliaceae) is a commonly used herbal medicine in many Asian countries. It is also used as a popular dietary supplement in recent years. There are two kinds of commercial ginseng products, including ginseng and red ginseng. Red ginseng is produced by steaming raw ginseng. Ginseng saponins, also termed as ginsenosides, are considered as the main bioactive components of ginseng. The pharmacological effects of these ginsenosides have been attributed to the biotransformation mediated by human intestinal bacteria. It has been recognized that red ginseng demonstrates more effective in pharmacological activities than ginseng in some notable respects. The differences in the bioactivities of ginseng and red ginseng may due to the changes of chemical constituents that occurred during the steam-processing. With the process of steaming or heating, the polar ginseng saponins were decreased, whereas the less polar ginseng saponins were increased. On the other hand, as the first example of ginseng saponins that found outside of the Araliaceae family, Gynostemma pentaphyllum contains more than 100 different gypenosides. Gypenosides are structurally identical to known ginsenosides, such as Rd, Rb1, Rb3, F2, Rc, Rg3, as well as malonylginsenosides Rb1 and Rd, make up around 25% of the total gynosaponins in Gp. The saponins isolated from notoginseng include notoginsenosides, ginsenosides and gypenosides. Among these saponins, ginsenoside Rg1, Rb1, Rd, and notoginsenoside R1 are considered to be the main constituents in Radix Notoginseng. The differential response of fecal microflora to these four different plant based saponins can be attributed to their different chemical constitutes. Among the four plant based saponins, red ginseng and ginseng saponins treated mice presented relatively similar profiles of microbial composition but still with distinguished changes in a time dependent manner. It is reasonable to suppose that this phenomenon may be associated with the similarities and differences in the chemical profiles between red ginseng and ginseng. The fecal metabolic profiles were also similar in red ginseng and ginseng saponins treated mice. On the other hand, the fecal microflora composition as well as the fecal metabolites in notoginseng saponins treated mice was closer to the control mice than other plant based saponins treated mice. All these findings can be a result of different ingested saponins-caused different microbial alteration followed by corresponding changes in the fecal metabolites.

Accumulating evidence indicates that the gut microflora play an important role in the development of obesity, diabetes, cancer, chronic liver disease and inflammatory bowel disease, etc. Among these diseases, the association between the gut microbiota and metabolic disorders has been well studied. Abnormal microbial composition has been identified as a key regulator in metabolic disorders. For examples, a shift in the ratio between Firmicutes and Bacteroidetes has been linked to obesity. It is reasonable to suggest that some diseases will affect the composition of the gut microbiota and regulation in the microbiota can contribute to the treatment of disease. Interestingly, we found that the level of Bacteroides, a major genus within the phylum Bacteroidetes, can be increased by the treatment of plant based saponins, including Gp, notoginseng, red ginseng and ginseng saponins. Although this finding was observed in the normal mice, it still provided a hint that plant based saponins can play a role in modulating the gut microbiota in the diseases with a shift ratio of Firmicutes and Bacteroidetes. Current strategies for manipulating the microbiota mainly include probiotics, prebiotics and synbiotics (a combination of probiotics and prebiotics). Some functional food and herbal medicines may also have the similar effects. Here, we found Gp and notoginseng saponins showed much more effect on the beneficial bacteria, including Bifidobacterium pp. and Lactobacillus pp. Prebiotics are known as non-digestible food ingredients that can enhance the growth or activity of beneficial microbes, such as oligofructose and inulin. It seemed that the two plant based saponins, Gp and notoginseng saponins, can also function as prebiotics, which will stimulate the growth of helpful bacteria and be conducive to good health. As is known, compound K is more effective than ginsenoside Rb1 in certain aspects such as anti-tumor, anti-inflammatory, and anti-allergic activities. The populations of Bacteroides and Bifidobacterium have been suggested to potently metabolize ginsenoside Rb1 to compound K. Gut microbial variations play an important role in drug metabolism, efficacy and toxicity in the host and gut microbiota have already been suggested to be taken into consideration in personalized health care in future. Different microbial composition may cause different drug response. Modulation of the gut microbiota may increase the capacity of drug metabolism.

Saponins from Gynostemma pentaphyllum (Gp) and the Effects on Tumor

Gynostemma pentaphyllum (Gp) is consumed as an herbal tea as well as folk medicine that was well documented in the Compendium of Materia Medica in China dated back to 16th Century for treating various symptoms, including cancer. The main active components in Gp are triterpenoid saponins named gypenosides. Our current finding demonstrated that treatment with Gp total saponins (GpS) exerts anti-cancer effects in xenograft nude mice. In this example, the gut microbial compositions between the normal and the tumor-bearing nude mice are compared, and then how GpS treatment would shape the composition of the gut microflora in both healthy and tumor-bearing animals are investigated.

Materials and Methods

Animals and Treatments

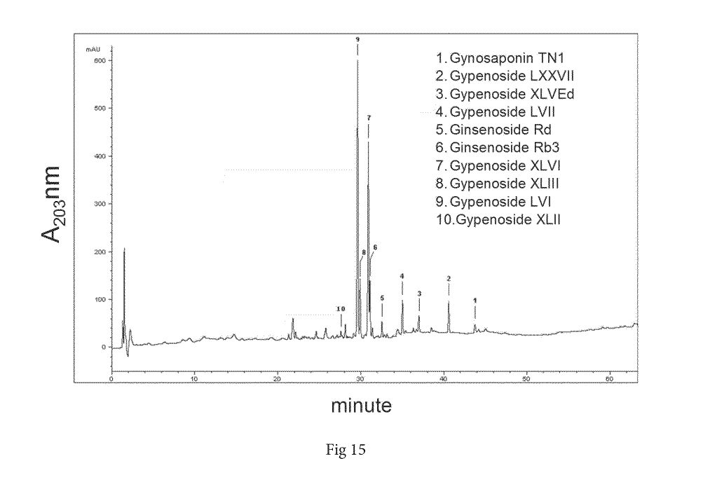

Animal welfare and experimental procedures were performed strictly in accordance with the care and use of laboratory animals. All procedures were approved by the University Ethics Review Committee for animal research. The athymic nude mice (BALB/c-nu/nu) were purchased from Chinese University of Hong Kong and maintained in IVC cages, on a 12-h light/dark cycle. Xenograft was done by injecting 106 R6/GFP-Ras transformed cells into the right flank of each 7-8 weeks old mice. The Rat6/GFP-Ras cell line is a transformed clonal cell line established from a transformed focus derived from R6 rat fibroblast cultures transfected by a GFP-tagged ras oncogene vector in our laboratory. The total saponins (GpS), extracted from the aerial parts of Gynostemma pentaphyllum, was purchased from the Hauduo Natural Products (Guangzhou, China). Authentication and chemical profiling of each batch were monitored for qualitative control according to Wu P K, Tai C S, Choi C Y, Tsim W K, Zhou H, Liu X et al., (2011). Chemical and DNA authentication of taste variants of Gynostemma pentaphyllum herbal tea. Food Chemistry 128: 70-80 (see FIG. 15). GpS was dissolved in 0.5% carboxymethyl cellulose (CMC) at 50 mg/ml. Single dose of GpS at 750 mg/kg or solvent control was given daily by gavage, started the second day after the implant of GFP-Ras cells. For the antibiotic intervention experiment, mice were pretreated with antibiotic (Penicillin/Streptomycin, 10 mg/ml; GIBCO 15140) or saline (control) by gavage (twice/day, total 700 μl/mouse/day) for 5 days, followed by implantation of 106 GFP-Ras transformed cells as described above.

Fecal Samples Collection

For experimental animals, fecal samples were collected (8:00-10:00 a.m.) at day 0 (before xenograft), and 5 days and 10 days after GpS treatment. For the antibiotic intervention, fecal samples were collected one day before and 5 days after antibiotic intervention, then 5 days and 10 days after GpS treatment. All fecal samples were immediately stored at −20° C. and kept for later DNA extraction.

Bacterial Genomic DNA Extraction from Fecal Samples

Total genomic DNA was isolated from fecal samples as described in Kong J, Li X B, Wu C F (2006). A molecular Biological Method for Screening and Evaluating the Traditional Chinese Medicine Used in Pi-deficiency Therapy Involving Intestinal microflora. Asian Journal of Traditional Medicines 1: 1-6 and McCracken V J, Simpson J M, Mackie R I, Gaskins H R (2001). Molecular ecological analysis of dietary and antibiotic-induced alterations of the mouse intestinal microbiota. The Journal of nutrition 131: 1862-1870, with slight modification. 0.1 g of fecal samples were vortexed in 4 ml sterile PBS (pH7.4) for 5 min, then centrifuged at 40×g for 8 min to collect the upper phase containing the bacteria. After repeating this procedure once, the supernatant was centrifuged at 2000×g for 8 min. The supernatant was discarded and the bacterial pellets were then washed twice with PBS for DNA isolation. DNA concentration was determined by NanoDrop 1000 spectrophotometry.

ERIC (Enterobacterial Repetitive Intergenic Consensus)-PCR

ERIC sequences are non-coding, highly conserved intergenic repeated sequences that reside in the genome of various bacterial species in addition to enterobacteria. ERIC-PCR was used to profile the gut microbiome using fecal genomic DNA as the template and a pair of ERIC specific primer sequences: ERIC 1R (5′-ATGTAAGCTCCTGGGGATTCAC-3′) and ERIC 2 (5′-AAGTAAGTGACTGGGGTGAGCG-3′). The PCR reaction was optimized and determined with orthogonal array design. A 25 μl reaction mixture containing 5 μl 5×PCR reaction buffer, 250 μM dNTP, 2 mM Mg2+, 0.4 μM primers, 1.5 unit Hotstart Taq polymerase, and 50 ng fecal genomic DNA. PCR was performed under the following conditions: 94′IC for 5 min, followed by 35 cycles of 95′IC for 50 seconds, 49′C for 30 seconds, 46′IC for 30 seconds, and 72′C for 3 min; and then a final extension at 72IC for 9 min. 10 μl of each PCR product was loaded into a 2% (w/v) agarose gel containing 0.5 μg/ml ethidium bromide and run for 40 min at 100 V. A DNA ladder (0.1-10.0 kb) was used as DNA size marker (NEB, N3200). Agarose gels were photographed using a Gel Doc™ XR+ System.

Data Analysis of ERIC-PCR Fingerprints

Partial least squares discriminant analysis (PLS-DA) was performed to analyze the dynamic changes of microflora composition of experimental groups. Based on the distance and the intensity of each DNA bands, the banding patterns of ERIC-PCR products separated on the gel were digitized by Image Lab 3.0 system (Bio-Rad) and performed PLS-DA analysis using SIMCA-P 12.0 tool. The Correlation coefficient was calculated and used to assess the correlation between two samples using the CORREL function in Microsoft Office Excel 2003.

16S rRNA Pyrosequencing of Fecal DNA Samples

PCR was performed for each sample in a final reaction volume of 25 ul comprising 0.1-2 μl DNA, 300 nM of each primer (563F and 1064R of 16S rRNA gene), 2.5 μl of 10× Expand High Fidelity buffer (Roche), 200 μM PCR Grade Nucleotide Mix, and 2.6 units of Expand High Fidelity Enzyme mix (Roche) with the reaction volume adjusted using milli-Q H2O. The forward primer of each reaction had a unique 11-nt barcode to enable demultiplexing of reads post-sequencing. The PCR conditions were conducted with an initial denaturation at 94° C. for 2 min followed by 35 cycles of 94° C. for 15 s, 58° C. for 20 s, and 72° C. for 1 min. Finally, an elongation reaction for 7 min at 72° C. was performed followed by cooling at 4° C. until collection. Amplicon sizes were confirmed on 1% agarose gel and purified with PureLink Quick Gel Extraction Kit (Life Technologies). Amplicon libraries were quantified with Quant-iT PicoGreen dsDNA Assay Kit (Life Technologies) using FLUOstar OPTIMA F fluorometer (BMG Labtech GmbH, Offenburg, Germany) and visually assessed using the FlashGel System (Lonza Group Ltd., Basel, Switzerland). Emulsion-PCR and pyrosequencing using titanium chemistry on the GS Junior System (454 Life Sciences Corp., Branford, Conn., USA) was carried out as detailed by the manufacturer.

Denoising and Analysis of Pyrosequencing Data

Pyrosequencing data were processed and analyzed using the Quantitative Insights Into Microbial Ecology software (QIIME version 1.5.0), available at http://qiime.sourceforge.net/. Denoising of raw sequences was performed to reduce the amount of erroneous operational taxonomic units (OTUs). Sequences were removed if they were <200 or >1000 nucleotides, with quality score below 25, contained primer mismatches or uncorrectable barcodes, or had a homopolymer run or ambiguous bases in excess of 6. The denoised sequences were assigned to OTUs with a 97% identity threshold, and the most abundant sequence from each OTU was selected as a representative sequence showing up in that OTU. Taxonomy was assigned to OTUs by using the Basic Local Alignment Search Tool (BLAST) for each representative sequence. For tree-based analyses, PyNAST was used to align these representative sequences of each OTU, and FastTree algorithm was used to build a phylogenetic tree. The differences in overall community composition between compared samples were determined using the unweighted UniFrac metric. Linear discriminant analysis (LDA) effect size (LEfSe) method was used to evaluate the key phylotypes responsible for the observed differences between microbial communities. OTU network was generated by QIIME and visualized with Cytoscape. Shannon-Weiner diversity index (H′) was used to evaluate the diversity of microbial communities. Venn diagram was used to figure out the unique and shared taxa between microbial communities.

Statistical Analysis

The data obtained are presented as means±SEM., and statistical comparisons were performed using one-way ANOVA followed by Student's t-test at P values of <0.01(**) or <0.05(*).

Results

A Significant Shift in the Gut Microbiota of the Xenograft Animals

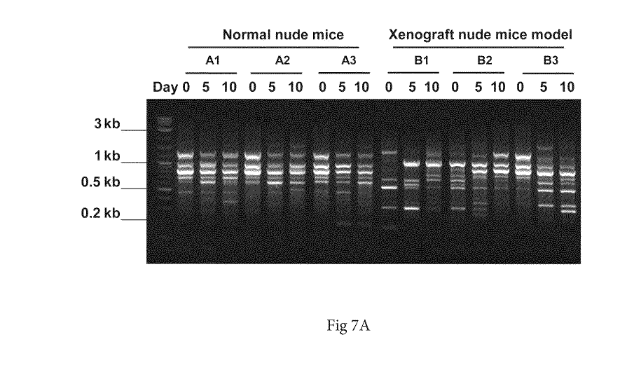

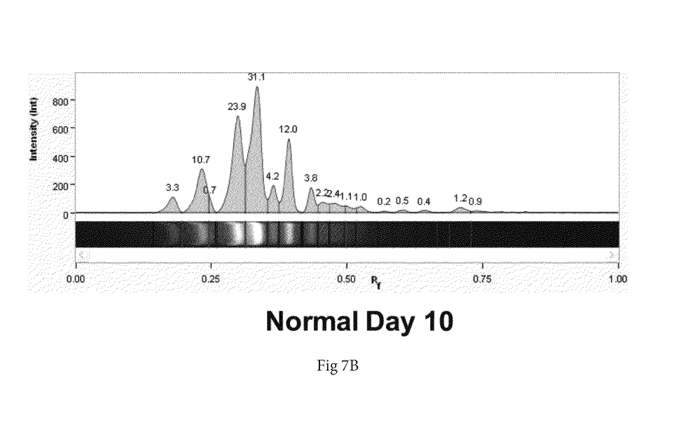

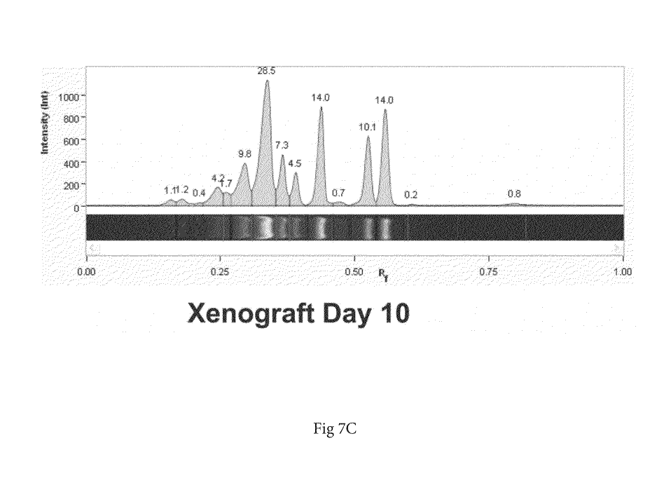

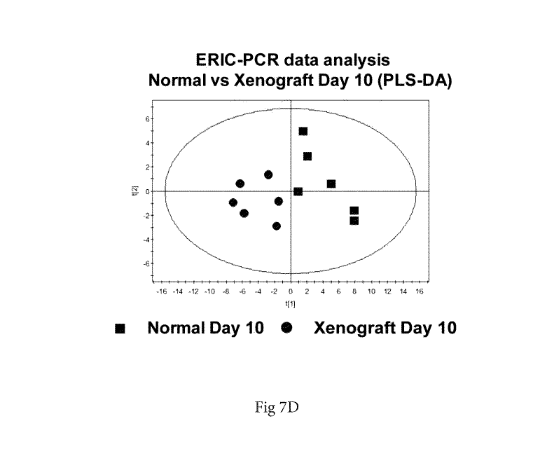

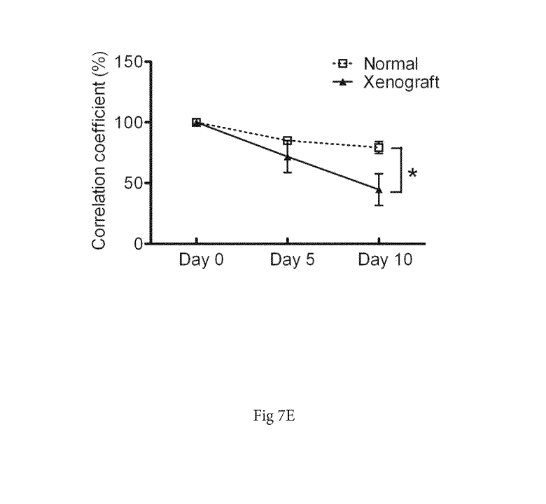

Microflora of healthy individual contains a balanced composition. In the diseased state of the host, there is a shift in the composition of the microflora, such as a reduction in the symbionts or an increase in the pathobionts. To investigate whether tumor xenograft would induce shift in gut microbiota, nude mice with and without xenograft were used as the animal models. Fecal samples were collected from the experimental animals for microbial DNA preparation and used for ERIC-PCR analysis of fecal microflora profile. As shown in FIG. 7A, similar banding patterns were observed among individual mice from the normal group throughout the experimental period, while obvious alterations in banding pattern were appeared among the xenograft-mice. The banding patterns were then digitized by Image Lab 3.0 system (Bio-Rad) and performed PLS-DA analysis (FIGS. 7B & 7C). The fecal microbiota of the normal and the tumor bearing mice were clearly separated in the PLS-DA plot (FIG. 7D) and in the correlation coefficient plot for divergent analysis (FIG. 7E). These findings suggest that the microflora likely maintained different patterns between normal and tumor-bearing mice. Tumor growth can induce the shift in the microflora composition.

GpS Inhibited Tumor Growth and Concurrently Regulated Microflora Composition

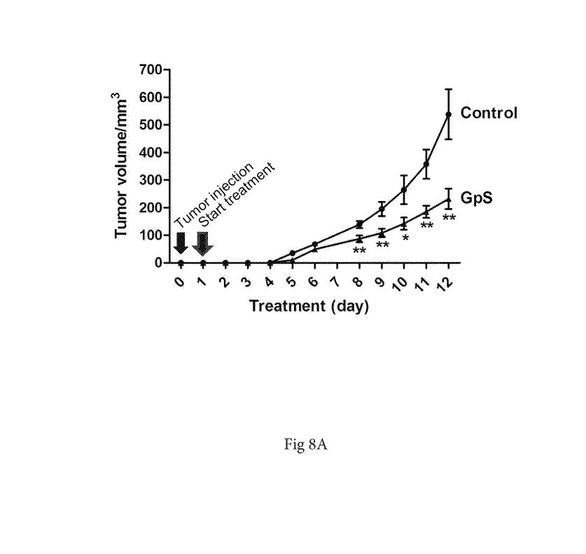

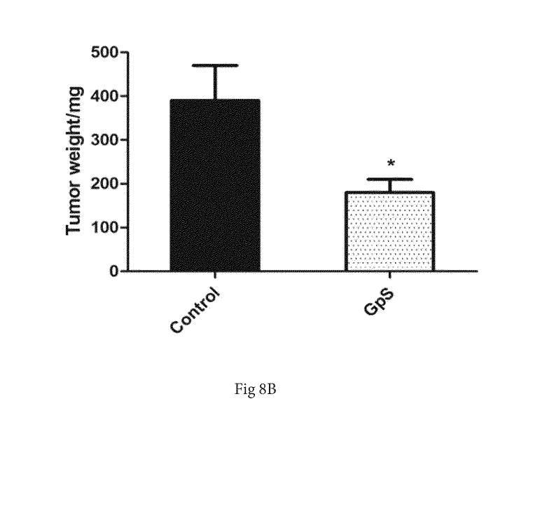

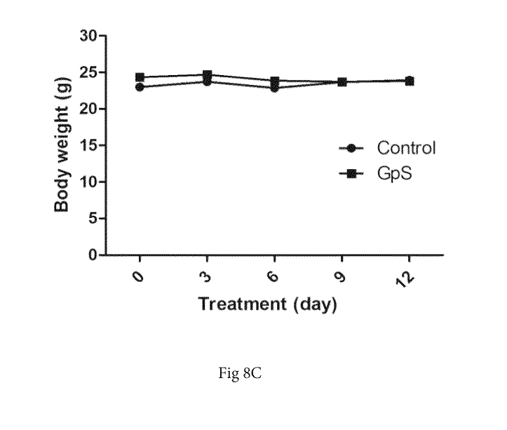

To test the effect of GpS on tumor growth, GFP-Ras cells (106) were subcutaneously injected into the right flank of each 6-8 weeks old nude mice. Tumor was measured with an electronic caliper in a blinded manner daily and tumor volume is calculated using the formula, (length×width2)/2. The control mice were injected with same volume of PBS solution. Single daily dose of GpS at 750 mg/kg or vehicle (0.5% CMC) by gavage started the second day after the implant of GFP-Ras cells and carried out for 12 days. The tumor volume and tumor weight of GpS-treated group reduced by 60% and 50% compared to the untreated group (FIGS. 8A & 8B). No weight loss in the treatment group was observed (FIG. 8C).

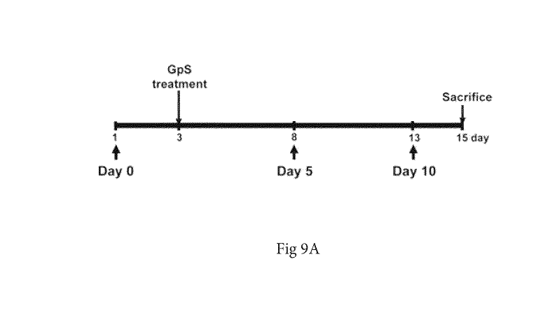

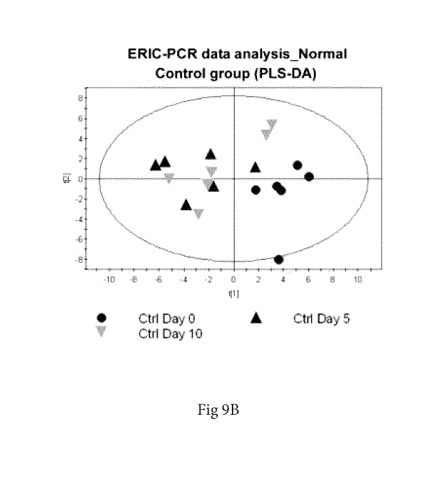

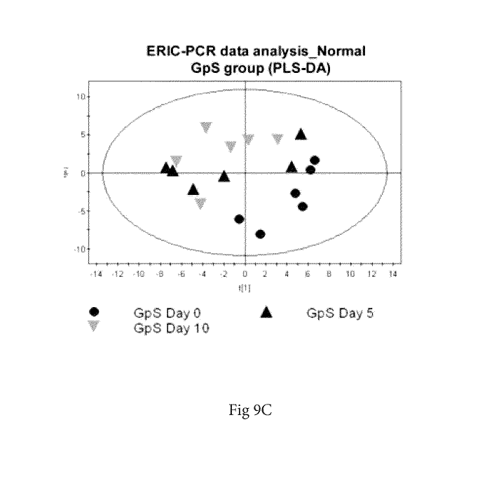

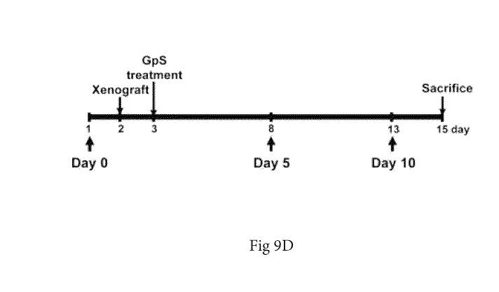

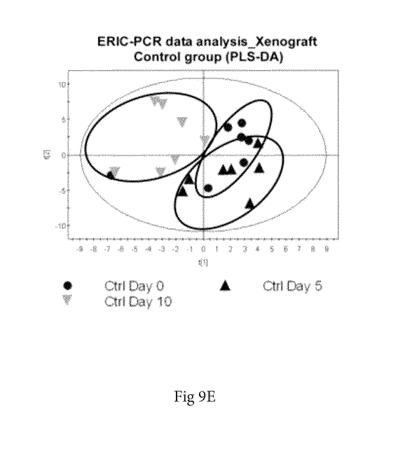

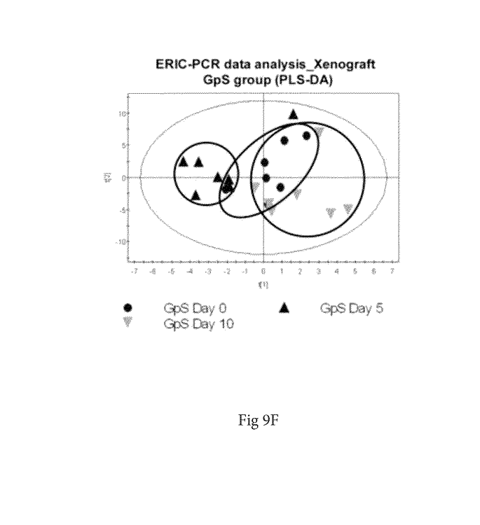

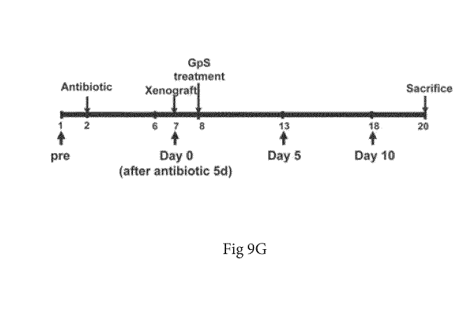

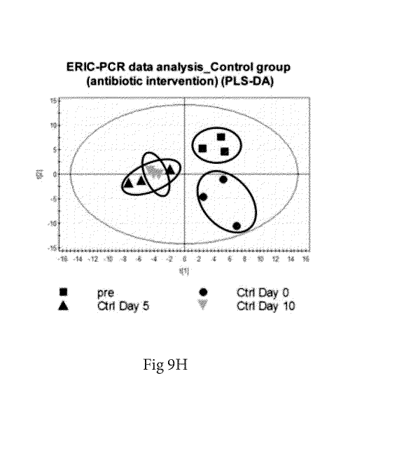

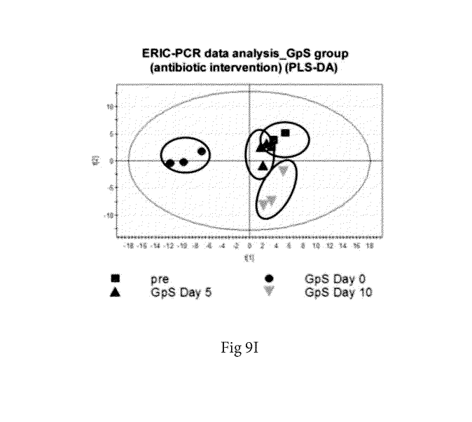

To investigate how GpS would modulate the gut microflora in the normal and xenograft nude mice, fecal samples were collected from four experimental groups, i.e. the normal group with and without GpS treatment; and the xenograft group with and without GpS treatment at Day 0, Day 5 and Day 10 as described in the treatment schemes (FIGS. 9A & 9D). Genomic DNA isolated from the fecal samples were analyzed by ERIC-PCR. The PLS-DA plots, based on the ERIC-PCR banding patterns, displayed a rather random modification of microbiota between Day 0 vs Day 5/10 time points in both GpS-treated or control normal mice (FIGS. 9B & 9C). In the xenograft mice, on the other hand, fecal microbiota from Day 0, Day 5 and Day 10 groups seems to clustering together within each group, yet drifting apart from Day 0 time point (FIG. 9E). Interestingly, upon GpS treatment, the microbiota community of Day 10 group was drifting back to the non-tumor stage and aligned mostly with Day 0 microbiota (FIG. 9F), which was not observed in the non-treatment groups (FIG. 9E). Such shift of microbiota composition induced by GpS was also observed in xenograft nude mice treated with antibiotic prior to tumor injection and GpS treatment (FIG. 9H vs 9I). Pretreatment with antibiotic helped to synchronize, but did not alter the gut microflora in the experimental mice.

16S Pyrosequencing Further Revealed the Different Microbial Communities Between the Normal and Xenograft Nude Mice

To obtain more comprehensive information of the gut microbial communities in nude mice, we performed 16S rRNA pyrosequencing on the fecal DNA obtained from the Day 10 time point of normal and xenograft nude mice, with and without GpS treatment described in the experiment showed in FIG. 9A-9I (3 fecal samples per group, a total of 12 samples). A total of 147128 reads that passed quality control were produced in this study, with an average of 12261 sequences per sample. 399 distinct operational taxonomic units (OTUs) were determined after denoising using QIIME method according to Caporaso J G, Kuczynski J, Stombaugh J, Bittinger K, Bushman F D, Costello E K et al., (2010). QIIME allows analysis of high-throughput community sequencing data. Nature methods 7: 335-336.

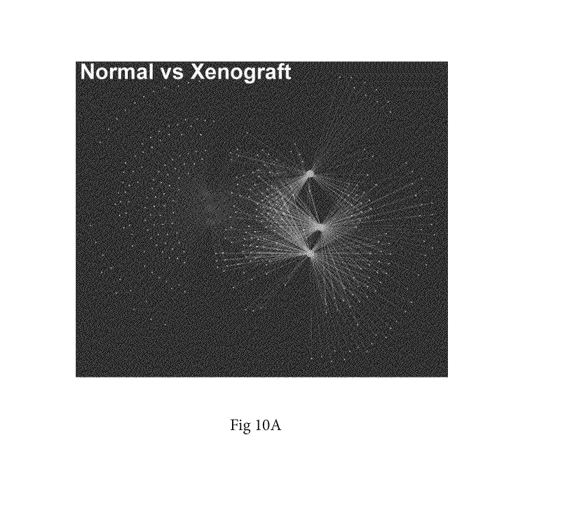

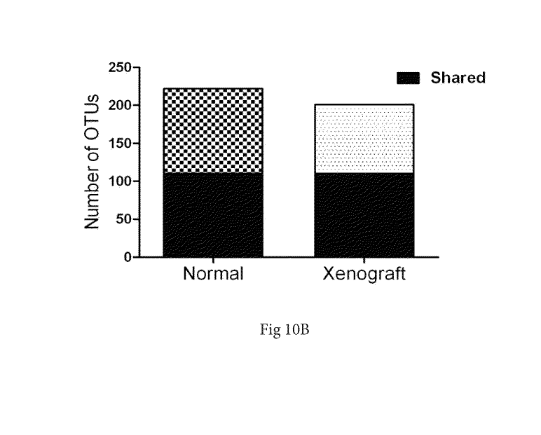

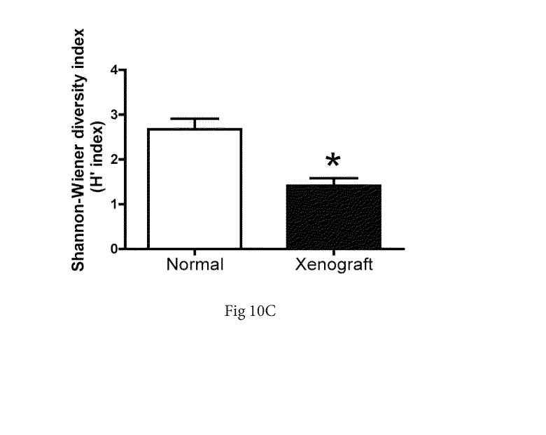

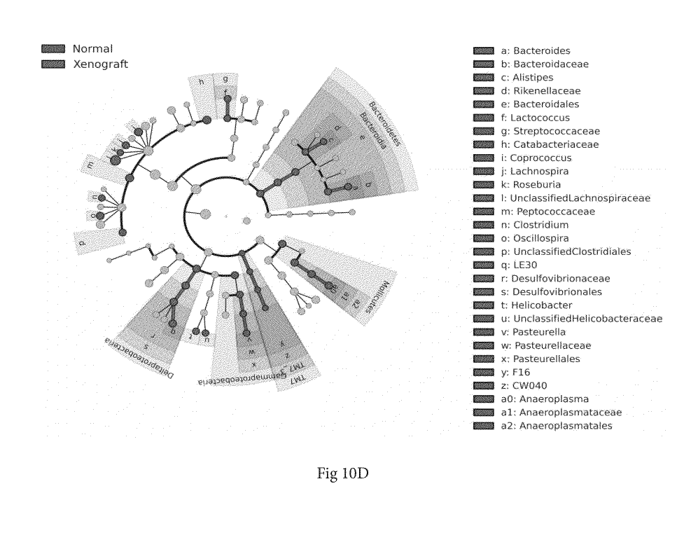

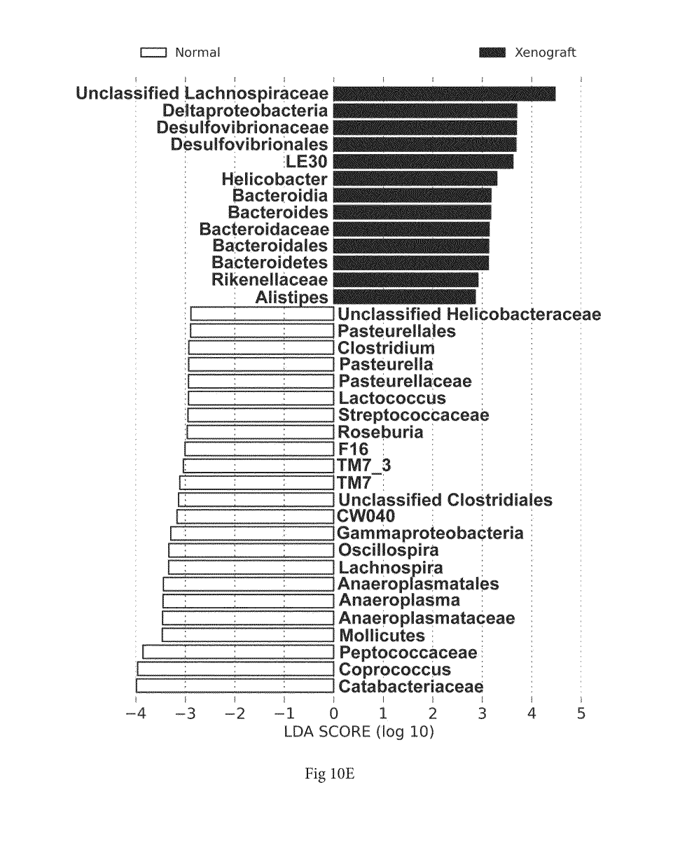

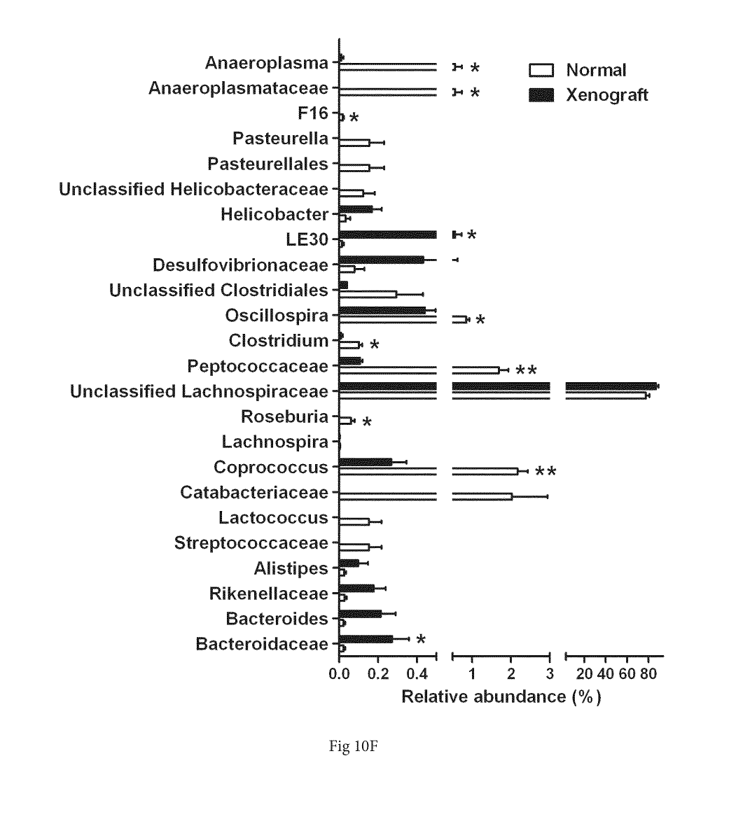

Denoised OTUs derived from the normal and xenograft mice were first collected and OTU network analysis was performed to generate an image of overall clustering of the test samples. As discussed in the previous session, tumor progression is likely to cause the separation in fecal microbiome between normal and xenograft nude mice. Similar finding was also observed in the OTU network analysis (FIG. 10A). By comparing the number of OTUs in nude mice with or without xenografted tumors, we found only 110 OTUs were shared. 112 and 91 unique OTUs can be found in normal and xenograft nude mice, respectively (FIG. 10B). In contrast to normal nude mice, reduced microbial diversity was found in xenograft nude mice based on the calculated Shannon-Weiner diversity index (FIG. 10C).

The 16S pyrosequencing data was then analysed using the Linear discriminant analysis (LDA) effect size (LEfSe) method to identify the key phylotypes responsible for the differences in fecal microbial communities between the normal and the xenograft nude mice. As shown in FIG. 10D, the taxonomic distribution of fecal microbiota between the normal and the tumor-bearing animals varied significantly at all taxonomic levels. At the phylum level, the most differentially abundant bacterial taxon in the feces of normal mice was TM7, whereas xenograft mice were overrepresented by Bacteroidetes (FIG. 10D-10F). Data showed that tumor-bearing nude mice harbored a fecal microbiota relatively enriched in Deltaproteobacteria but depleted in Gammaproteobacteria, both taxa are under Gram-Proteobacteria. Mollicutes, which is under the phylum of Tenericutes, was also underrepresented in xenograft nude mice (FIG. 10D). The histogram of the linear discriminant analysis (LDA) score (FIG. 10E) and the relative abundance score (FIG. 10F) showed the statistically and biologically differential clades appeared in the normal and xenograft nude mice. It is of worthy mentioned that although Firmicutes was not identified as the differentially abundant phylum, the clades under this phylum, such as Catabacteriaceae, Peptococcaceae, Coprococcus were particularly enriched in normal, but not in the xenograft mice (FIG. 10F).

GpS Treatment Significantly Altered Fecal Microbiota of Normal and Xenograft Nude Mice

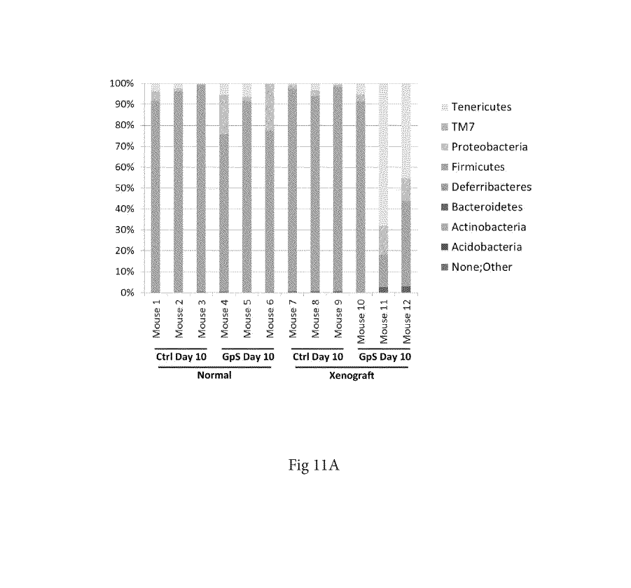

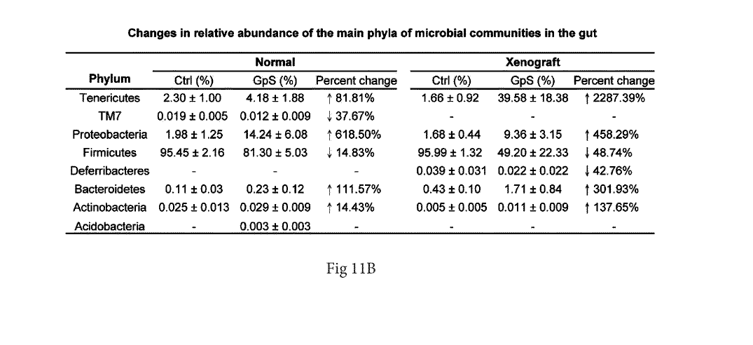

The 16S pyrosequencing data demonstrated that GpS treatment caused alteration of the relative abundance of dominant taxa in fecal microbiota both at the phylum (FIGS. 11A & 11B). However, the alteration induced by GpS is more prominent in the xenograft than the normal mice. Within the three prominent phyla, mice treated with GpS, compared to the untreated, exhibited relatively lower abundance of Firmicutes (from 95.45 down to 81.30%), and higher abundance of Tenericutes (from 2.3 to 4.18%), Proteobacteria (from 1.98 to 14.24%) as well as Bacteroidetes (0.11 to 0.23%) (FIG. 11B). In the xenograft mice, GpS treatment markedly reduced the Firmicutes (from 95.99% down to 49.2%), in the meantime, it increased substantially the relative abundance of Tenericutes (from 1.66% to 39.58%) and Proteobacteria (from 1.68 to 9.36%) (FIG. 11B). Although the Bacteroidetes levels are relatively low abundance, we observed a 3-fold higher relative abundance of Bacteroidetes in GpS treated xenograft nude mice than that in controls (FIG. 11B).

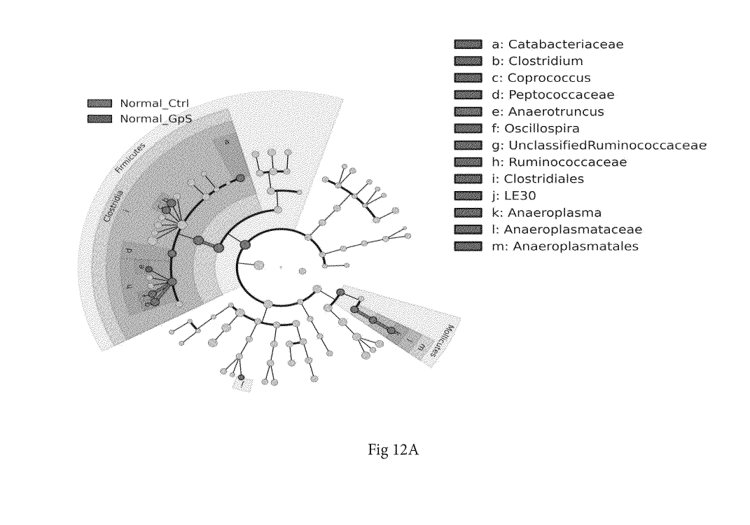

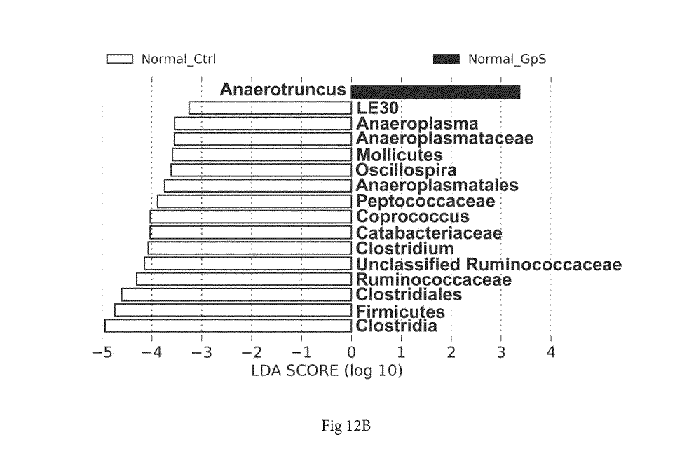

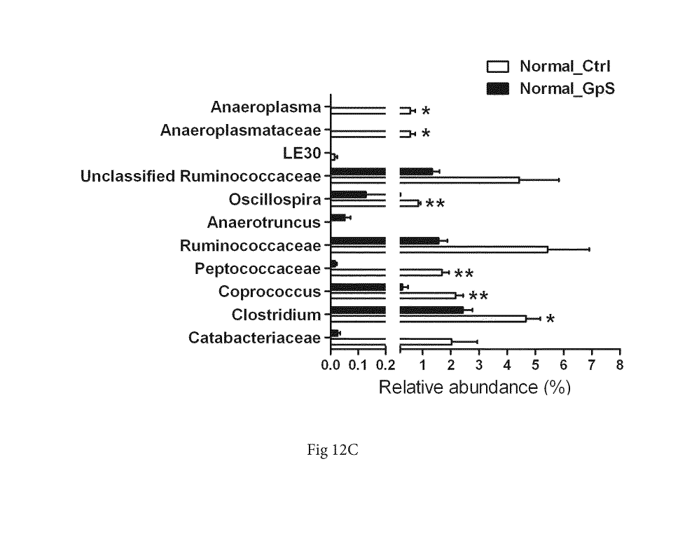

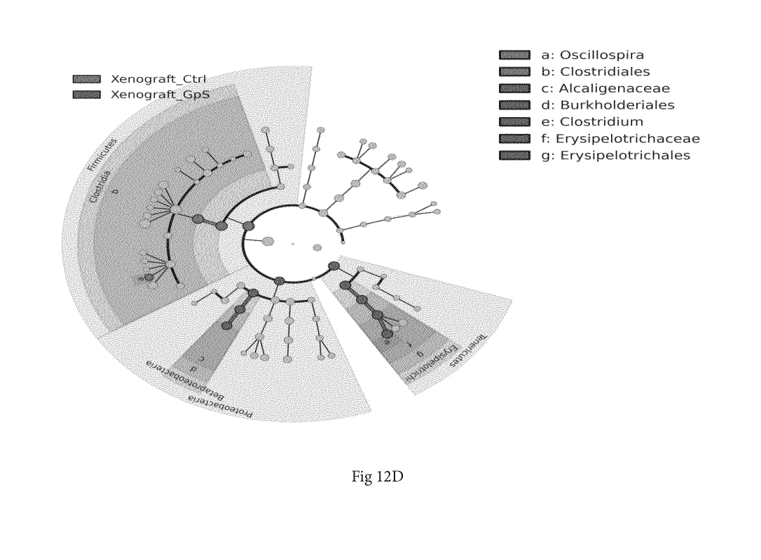

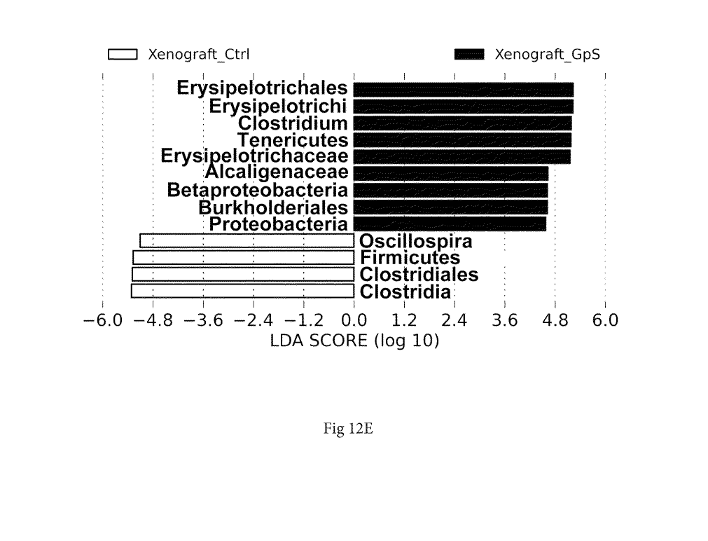

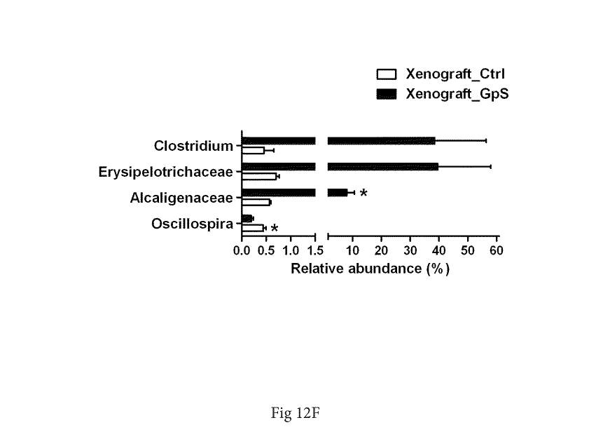

We further analyzed the differential microbial phylogenic structures of normal and xenograft nude mice responding to GpS treatment by LEfSe tool. The taxonomic representations of the analysis are displayed as cladograms in FIG. 12A-12F. In normal nude mice, two classes were identified as the differentially abundant bacterial taxa, including Clostridia and Mollicutes (FIG. 12A). Clostridia was identified with a very high LDA score (approximately five orders of magnitude, FIG. 12B), reflecting marked abundance in normal mice (mean 94.98%) and consistently relatively low abundance in GpS-treated mice (mean 80.33%). Within Clostridia, the families such as Catabacteriaceae, Peptococcaceae and Ruminococcaceae and the genus, such as Clostridium, Coprococcus, Oscillospira were all found enriched in normal mice relative to the GpS-treated mice. In addition, the lineages of Mollicutes, the class under Tenericutes, including Anaeroplasmatales, Anaeroplasmataceae and Anaeroplasma were all the differentially abundant clades in the normal group (FIG. 12A). Based on the relative abundance score (%), Anaerotruncus, the genus under Clostridia, was the only differentially abundant taxon detected in the treated mice, while all other families and genera were all significantly lower in relative abundance compared to untreated mice (FIG. 12C).

In the xenograft nude mice, the three dominant phyla: Firmicutes, Proteobacteria and Tenericutes showed differential responses to GpS treatment. The major components contributing to these three distinguished phyla were the following classes: Clostridia (uncer Firmicutes) (95.84% vs. 49.08%), Betaproteobacteria (under Proteobacteria) (0.81% vs. 8.01%) and Erysipelotrichi (under Tenercutes) (1.65% vs. 39.58%) between the control and the GpS-treated mice (FIG. 12D). Differences of bacterial community structure between the control and the treatment mice were also demonstrated in the phylogenetic structure within individual lineages. In the GpS-treated xenograft mice, there were two notable lineages showing relatively high abundance with the greatest differences compared to the controls. One was the Proteobacteria-Betaproteobacteria-Burkholderiales-Alicaligenaceae lineage. The other was Tenercutes-Erysipelotrichi-Erysipelotrichales-Clostridium lineage (FIG. 12D). At the family level of these two particular lineages, we found that Alcaligenaceae (0.81% vs. 8.01%) and Erysipelotrichaceae (1.65% vs. 39.58%) were the main differentially abundant clades in the GpS-treated mice. At genus level, Clostridium presented greatest variations with over five orders of magnitude difference in abundance between the two groups (FIG. 12E). It constituted less than 0.5% of total bacteria in controls, however, it was much more prevalent in the GpS-treated mice (mean 38.48%).

Identification of the Unique Bacterial Families Associated with Different Treatment Groups

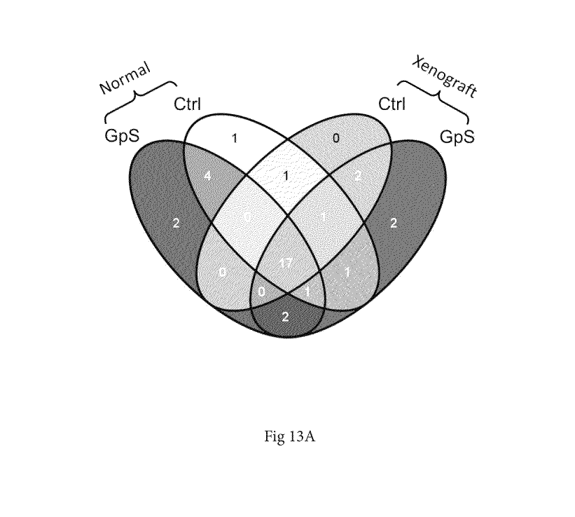

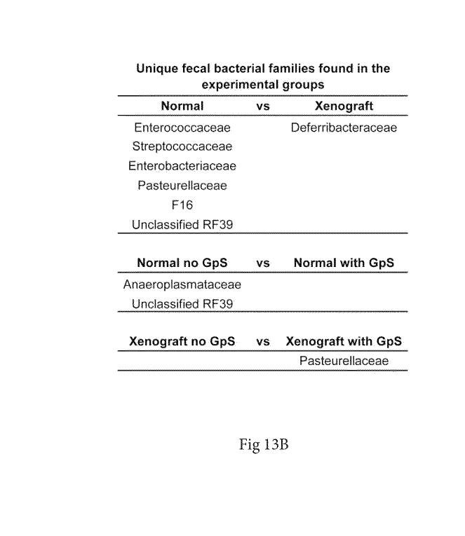

To identify taxa that are unique to different treatment groups, we compared the unique and shared bacterial families by Venn Diagram. As shown in FIG. 13A, there were 7 and 2 unique bacterial families found in the normal and xenograft mice, respectively, while 19 bacterial families were overlapped between the two groups. When compared the microbial communities in the normal nude mice with or without GpS treatment, we found 22 bacterial families were shared, whereas control and GpS-treated groups each exclusively harbored 4 different families. On the other hand, in xenograft nude mice, 20 bacterial families were detected in both controls and GpS-treated mice. Only one unique bacterial family was found in controls while six unique families were found in the GpS-treated xenograft mice. These unique bacterial families with a mean relative abundance >0.01% were listed in FIG. 13B. Deferribacteraceae (under Deferribacteres) was only detected in xenograft nude mice, while Enterococcaceae, and Streptococcaceae under Firmicutes, Enterobacteriaceae and Pasteurellaceae under Proteobacteria, and two unclassified F16 and RF39 were unique to normal individuals. Comparing the normal nude mice with or without GpS treatment, we found the unclassified RF39 and Anaeroplasmataceae (under Proteobacteria) were absent from the GpS-treated individuals. Another interesting finding was that Pasteurellaceae, which was depleted in xenograft nude mice, was presented in the GpS-treated tumor bearing mice.

Identification of Bacterial Species Altered Upon GpS Treatment

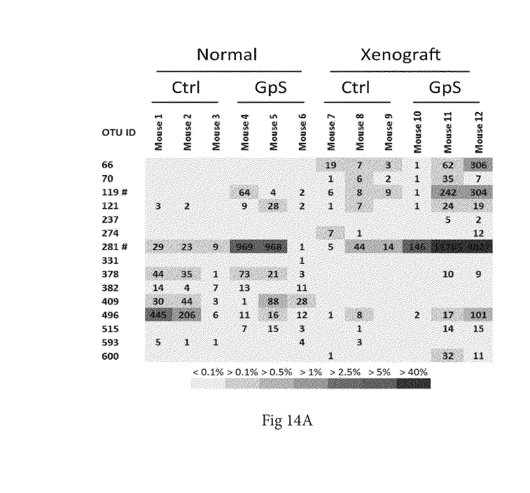

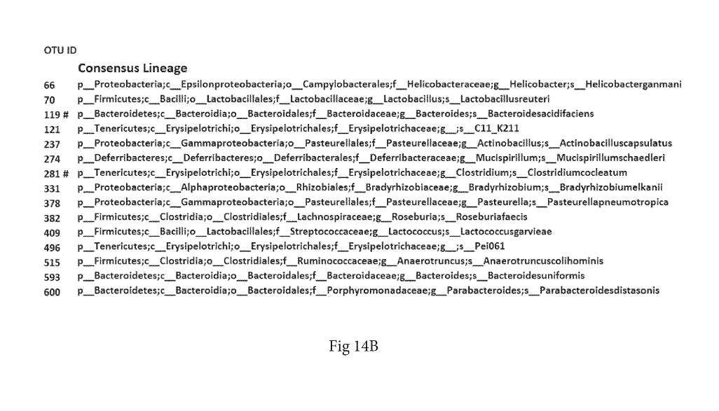

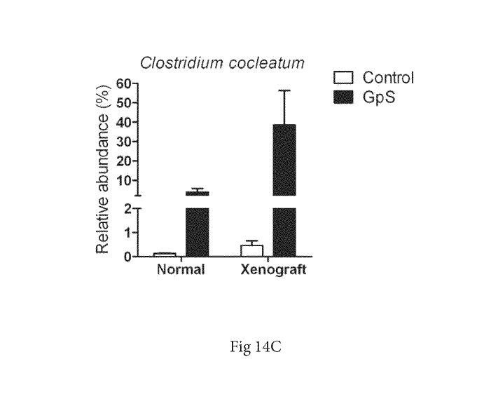

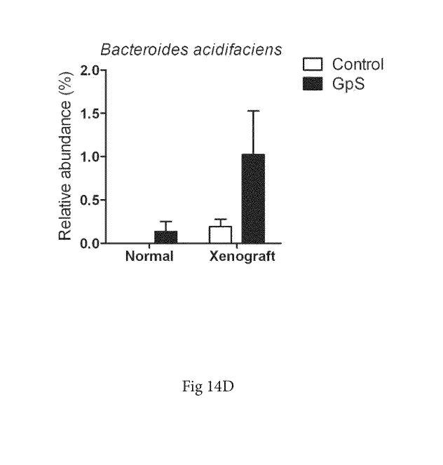

Based on the consensus lineage map of OTUs generated by QIIME software, most of the bacteria identification can be down to the genus level, while few can be identified to the species level. For example, one OTU is related to Clostridium cocleatum, and another OTU is related to Bacteroides acidifaciens. Both species showed an increasing trend after GpS treatment in both normal and xenograft nude mice. Compared to the un-treated control, Clostridium cocleatum increased more than 28 fold (in relative abundance) in GpS-treated normal mice. In the xenograft groups, an 80 fold increment of relative abundance of Clostridium cocleatum in response to GpS treatment. The relative abundance of Bacteroides acidifaciens increased by 5 fold compared to the un-treatment controls (FIG. 14A-14D). The striking increase of Clostridium cocleatum appeared to be an important driver of fecal bacterial community structure in GpS-treated tumor bearing mice.

### DISCUSSION

The gut microflora are believed to shape intestinal immune response during health and disease. Host immune regulation in turn is also vital in shaping a normal microbiota; disturbance of host regulation creates a dysbiotic microbiota, which is characterized by an imbalanced microflora community. In addition, it is evidence that different dietary compounds would interact and affect the regional or temporal composition of the gut microbiota. Gp herbal tea, similar to the green tea, can be consumed as regular tea, it also has various medicinal functions including anti-cancer effect. The questions addressed in this example are two-fold: 1) How would gut microbiota response to dietary/medicinal saponins under healthy and diseased states? 2) As our data indicated, Gp sapoinins treatment can significantly reduce the size of xenograft tumor. Could there be a link between the tumor growth and the composition of gut microbiota? To address the questions, we employed ERIC-PCR and 16S pyrosequencing methods to systematically monitor the structural dynamics of fecal microbial communities in nude mice subjected to different treatments. PLS-DA plots of ERIC-PCR data revealed an observed correlation between changes in microbial composition and the disease phenotype. Pyrosequencing based LEfSe analysis, based on the pyrosequencing data, demonstrated that tumor xenograft can markedly modify gut microflora at various phylogenic levels. Normal nude mice are enriched with Firmicutes while xenograft mice are enriched with Bacteroidetes has identified key bacterial alterations between normal and xenograft nude mice, which may provide possible biomarkers used for detecting or monitoring cancer development. Meanwhile, we found a decline in microbial diversity occurred in tumor-bearing nude mice, which may be a byproduct of the cancer process. Likewise, the reduced microbial diversity can also be found in other diseases, such as inflammatory bowel disease and obesity. Our results hint at potential and plausible features of a cancer-induced dysbiotic microbiota. It is possible that tumor progression leads to dysregulation of the immune system, accounting for the alteration in microbiota.

As is known, ERIC-PCR is initially used to detect species under Enterobacteriaceae and Vibrionaceae families, including few top organisms such as Escherichia coli, Salmonella enterica, Yersinia pestis and Vibrio cholera. Later, Eric sequences are also found in the genome of various bacterial species as described in Delihas N (2007). Enterobacterial small mobile sequences carry open reading frames and are found intragenically—evolutionary implications for formation of new peptides. Gene regulation and systems biology 1: 191-205; Wang L, Jin Y, Zhao L, Pang X, Zhang X (2009). ERIC-PCR-based strain-specific detection of phenol-degrading bacteria in activated sludge of wastewater treatment systems. Letters in applied microbiology 49: 522-528 and Wilson L A, Sharp P M (2006). Enterobacterial repetitive intergenic consensus (ERIC) sequences in Escherichia coli: Evolution and implications for ERIC-PCR. Molecular biology and evolution 23: 1156-1168. To these microbial communities identified by ERIC-PCR, two interesting aspects were revealed. Firstly, tumor xenograft were able to alter the gut microbiota with a rather short period of time (FIG. 7A-7E). Secondly, GpS treatment can modulate the dysbiosis in tumor-bearing status and restore the microflora composition back to the non-tumor situation on the day of tumor cell injection (FIGS. 9F and 9I). Such shift was also observed in xenograft nude mice pretreated with antibiotic prior to tumor injection and GpS treatment (FIG. 9H vs 9I), but not observed in the normal mice obtained same GpS treatment. Whether this alteration of gut microbiota was a refection of regression of the tumor or a direct effect of GpS treatment is of interest for further investigation. During tumor progression, GpS treatment was likely to achieve balance of the microbial ecosystem by counteracting the alterations of these ERIC-PCR detected bacteria, such as Enterobacteriaceae family, which was also significantly more abundant in other disease model like colitis-susceptible I110−/− mice.

Subsequently, the fecal microbial communities were assessed and compared by 16S pyrosequencing to obtain a more comprehensive microbiota profile. At phylum level, Tenericutes, Proteobacteria and Bacteroidetes were more abundant in GpS-treated nude mice than in controls, whereas Firmicutes showed the opposite pattern, especially in tumor-bearing nude mice. A shift in the ratio between Firmicutes and Bacteroidetes has been reported in many other studies. It has been linked to many diseases, such as obesity. Based on the analysis of pyrosequencing data, we found that Bacteroidetes/Firmicutes ratio showed an increased trend after 10 days of GpS treatment in nude mice with xenografted tumors. On the other hand, we found GpS treatment can increase the relative abundance of Proteobacteria which are the major group of Gram-negative bacteria in the gut. The lipopolysaccharide (LPS) in the outer layer of the bacteria. LPS has been recognized in Goto S, Sakai S, Kera J, Suma Y, Soma G I, Takeuchi S (1996). Intradermal administration of lipopolysaccharide in treatment of human cancer. Cancer Immunol Immunother 42: 255-261 as a treatment for cancer by stimulating immune system. In our case, the increased Proteobacteria in GpS-treated mice may potentially enhance the secretion of LPS thus activate an immune response against tumors.

Pyrosequencing analysis also identified few species of bacteria upon GpS treatment. Clostridium cocleatum and Bacteroides acidifaciens were the two species showing increased trend in both normal and xenograft mice treated with GpS (FIGS. 14C & 14D), which have several well-documented beneficial effects. For example, study in Boureau H, Decre D, Carlier J P, Guichet C, Bourlioux P (1993). Identification of a Clostridium cocleatum strain involved in an anti-Clostridium difficile barrier effect and determination of its mucin-degrading enzymes. Research in microbiology 144: 405-410 indicated that strain of C. cocleatum can exert a protective barrier effect against the colonization the pathogenic Clostridium difficile in the gut and displayed multiple glucosidase activities that can involve in degrading the oligosaccharide chains of mucin in the digestive tract. C. cocleatum was significantly reduced in irritable bowel syndrome patients. In addition, C. cocleatum plays a role in the conversion of diglucoside, and have the de-glycosylation activity. Clostridium bacteria occupy a major fraction of mammalian gut microbiota and are responsible for promoting anti-inflammatory immune responses. It is concluded that C. cocleatum, which exhibited a striking increase in GpS-treated xenograft nude mice, contributed most to differentiate the microbial structures from the controls. Aside from the well-documented beneficial effects discussed above, C. cocleatum may take part in the metabolism of Gp saponins through glucosidase activities and have a role similar to symbionts.

Bacteroides acidifaciens was first isolated from the cecum of mice. B. acidifaciens and its closed relative, B. uniformis were found to be associated with the degradation of the isoflavone in human feces. Recent study in Yanagibashi T, Hosono A, Oyama A, Tsuda M, Suzuki A, Hachimura S et al., (2012). IgA production in the large intestine is modulated by a different mechanism than in the small intestine: Bacteroides acidifaciens promotes IgA production in the large intestine by inducing germinal center formation and increasing the number of IgA(+) B cells. Immunobiology demonstrated that B. acidifaciens promoted IgA production. It is reasonable to assume that the beneficial effects of Clostridium cocleatum and Bacteroides acidifaciens be potentially conducive to the anti-cancer effect of GpS. It is intriguing that the changes in gut microbiota observed in GpS-treated xenograft nude mice were more apparent than that in GpS-treated normal individuals. It seemed that the therapeutic effect of GpS was enhanced in some pathological conditions. One possible reason for this was that some pathological conditions generated a disturbed microbial system and GpS treatment can reverse this imbalance. The increase in these beneficial bacteria induced by GpS treatment, can function as symbionts and contribute to rebalancing the microbial ecosystem and exerting an inhibitory effect on tumor growth.

In conclusion, the present invention demonstrates how dietary saponins can exert regulating and balancing effects on the gut microbial ecosystem. The results indicated that tumor growth can impact on dynamics of the gut microbial ecosystem. At the same time, we also demonstrated that GpS treatment can alter the gut microflora composition, in particular boosting beneficial bacteria and then contributing to restore the dysbiosis back to eubiosis state.

Those skilled in the art will appreciate that the invention described herein is susceptible to variations and modifications other than those specifically described. If desired, the different functions discussed herein may be performed in a different order and/or concurrently with each other. Furthermore, if desired, one or more of the above-described functions may be optional or may be combined.

While the foregoing invention has been described with respect to various embodiments and examples, it is understood that other embodiments are within the scope of the present invention as expressed in the following claims and their equivalents. Moreover, the above specific examples are to be construed as merely illustrative, and not limitative of the reminder of the disclosure in any way whatsoever. Without further elaboration, it is believed that one skilled in the art can, based on the description herein, utilize the present invention to its fullest extent. All publications recited herein are hereby incorporated by reference in their entirety.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

Other definitions for selected terms used herein may be found within the detailed description of the invention and apply throughout. Unless otherwise defined, all other technical terms used herein have the same meaning as commonly understood to one of ordinary skill in the art to which the invention belongs.

1. Use of a composition comprising saponins extracted from plants for improving gut microbial ecosystem of a subject.

2. The use of the composition according to claim 1, wherein the plants comprising Gynostemma pentaphyllum (Gp), Panax pseudoginseng, Panax notoginseng and Panax ginseng.

3. The use of the composition according to claim 2, wherein the Panax ginseng is processed to comprise red ginseng.

4. The use of the composition according to claim 3, wherein the Panax ginseng is processed by steaming.

5. The use of the composition according to claim 2, where in the plants further comprising Radix Notoginseng of Panax pseudoginseng, Radix Notoginseng of Panax notoginseng and Radix Ginseng of Panax ginseng.

6. The use of the composition according to claim 1, wherein the saponins are of a range of concentration of about 500 mg/kg to 750 mg/kg in the composition.

7. The use of the composition according to claim 1, wherein the improvement to the gut microbial ecosystem comprising regulating and balancing the gut microbial ecosystem by increasing symbionts in the gut ecosystem of said subject.

8. The use of the composition according to claim 1, wherein said subject is a human.

9. The use of the composition according to claim 1, wherein the composition is used as prebiotics for improving the gut microbial ecosystem of a subject.

10. The use of the composition according to claim 1, wherein the improvement of the gut microbial ecosystem of a subject results in an inhibitory effect on tumor growth in said subject.
